(19) United States
(12) Patent Application Publication
(10) Pub. No.: US 2026/0136747 A1
(43) Pub. Date: May 14, 2026

(54) STRETCHABLE SEMICONDUCTOR FILM WITH BIOCOMPATIBILITY AND BIO-IMPLANTABLE ELECTRONIC DEVICE USING THE SAME

(71) Applicants: UNIVERSITY-INDUSTRY COOPERATION GROUP OF KYUNG HEE UNIVERSITY, Yongin-si (KR); RESEARCH & BUSINESS FOUNDATION SUNGKYUNKWAN UNIVERSITY, Suwon-si (KR)

(72) Inventors: Jin Young OH, Anyang-si (KR); Suk Ho BHANG, Suwon-si (KR); Ji Yu HYUN, Suwon-si (KR); Kyu Ho JUNG, Suwon-si (KR); Ji Seon AN, Suwon-si (KR)

(73) Assignees: UNIVERSITY-INDUSTRY COOPERATION GROUP OF KYUNG HEE UNIVERSITY, Yongin-si (KR); RESEARCH & BUSINESS FOUNDATION SUNGKYUNKWAN UNIVERSITY, Suwon-si (KR)

(21) Appl. No.: 19/318,970

(22) Filed: Sep. 4, 2025

(30) Foreign Application Priority Data
Nov. 14, 2024 (KR) 10-2024-0161768

(51) Int. Cl. H10K 10/46; A61L 31/10; C09D 119/00

(52) U.S. Cl. CPC H10K 10/488; A61L 31/10; C09D 119/006

(57) ABSTRACT

Disclosed are a stretchable semiconductor film having biocompatibility and an electronic device using the same. The stretchable semiconductor film having biocompatibility of the present disclosure includes a biocompatible elastomer matrix; and a semiconducting polymer of an interconnected semiconductor nanofiber network embedded in the elastomer matrix, wherein the semiconducting polymer of the interconnected semiconductor nanofiber network embedded in the elastomer matrix provides stretchability. The electronic device using the stretchable semiconductor film having biocompatibility of the present disclosure is characterized by using a bilayer electrode composed of gold and silver, providing excellent corrosion resistance in vivo.

### CROSS-REFERENCE TO RELATED APPLICATION

This application claims priority to Korean Patent Application No. 10-2024-0161768, filed on Nov. 14, 2024 in the Korean Intellectual Property Office, the disclosure of which is incorporated herein by reference.

### BACKGROUND OF THE DISCLOSURE

### Field of the Disclosure

The present disclosure relates to a stretchable semiconductor film having biocompatibility and an implantable bioelectronic device using the same.

### Description of the Related Art

Implantable bioelectronic devices have drawn significant attention in the biomedical field as they enable real-time personalized healthcare. These devices may allow for continuous monitoring of various electrophysiological signals and management of physical activities.

Commercial implantable medical devices, such as pacemakers, neurostimulators, and insulin pumps, are primarily composed of rigid inorganic materials like silicon-based semiconductors and metals.

While these materials are effective, their rigidity causes mechanical mismatches with soft biological tissues, which may lead over time to complications such as microdamage, inflammation, fibrosis, and necrosis.

To overcome these limitations, recent research has focused on developing soft, lightweight, and stretchable bioelectronic devices that may integrate seamlessly with human tissues.

In order to commercialize functionally-integrated implantable stretchable bioelectronic devices, the development of stretchable transistors, which are essential components for controlling stretchable current, is critical. In particular, it is important to impart stretchability to electrodes and semiconductor materials, which are typically made of rigid substances.

In addition, to be utilized as an implantable electronic device, it must be designed to maintain high biocompatibility and stability in physiological environments. Organic electrochemical transistors (OECTs) offer advantages such as compatibility with body electrolytes and stretchability; however, they still face challenges regarding limited functionality, circuit integration, and enhancement of device density.

Stretchable organic field-effect transistors (OFETs) are a promising alternative to organic electrochemical transistors for high-frequency signal processing. These devices are suitable for integration into bioelectronic circuits, as they may maintain mechanical flexibility and preserve electrical performance while utilizing conventional inorganic semiconductor-based technologies. Various strategies have been proposed for stretchable OFETs through nanoconfinement effects enabled by blending semiconductors with elastomers.

As a result, stretchable semiconductor materials have been realized through the blending of semiconductors and elastomers. However, since commercial elastomers have been used, their biocompatibility, which is essential for implantation, has not been verified, and thus they have not been developed into implantable materials.

In addition, due to the rigidity of metals, polymer conductors have been used as electrode materials; however, their biocompatibility has not been verified, and their electrical conductivity is lower than that of metallic materials. Therefore, there is a need to develop intrinsically stretchable and biocompatible electrode materials.

### RELATED ART DOCUMENTS

### Patent Documents

(Patent Document 1) Korean Patent Application Publication No. 10-2018-0113396, entitled “VERTICALLY AND LATERALLY PHASE-SEPARATED SEMICONDUCTING POLYMER/INSULATING POLYMER BLEND FILM AND ORGANIC FIELD-EFFECT TRANSISTOR COMPRISING THE SAME”

(Patent Document 2) Korean Patent No. 10-1543665, entitled “Biocompatible organic semiconductor films having organic-field effect transistor function and biocompatible organic thin film transistor using the same”

(Patent Document 3) Korean Patent No. 10-2329392, entitled “Composition for preparing organic insulator including aromatic polythiourea and crosslinking agent, organic insulator and thin film transistor prepared thereby”

### SUMMARY OF THE DISCLOSURE

Therefore, the present disclosure has been made in view of the above problems, and it is one object of the present disclosure to provide a semiconductor film with enhanced stretchability using a semiconducting polymer and an elastomer matrix.

It is another object of the present disclosure to provide a semiconductor film with improved mechanical and electrical properties.

It is still another object of the present disclosure to provide a semiconductor film for an implantable bioelectronic device, which has corrosion resistance, stretchability, and biocompatibility in biological environments.

It is still another object of the present disclosure to provide a stretchable electrode for an implantable bioelectronic device.

It is yet another object of the present disclosure to provide an implantable bioelectronic device with improved corrosion resistance, flexibility, stretchability, and biocompatibility in biological environments.

In accordance with an aspect of the present disclosure, the above and other objects can be accomplished by the provision of a stretchable semiconductor film having biocompatibility, including: an elastomer matrix having biocompatibility; and a semiconducting polymer of an interconnected semiconductor nanofiber network embedded in the elastomer matrix, wherein the interconnected semiconductor nanofiber network of the semiconducting polymer is formed through phase separation due to a difference in surface energy between the semiconducting polymer and the elastomer matrix during a mixing process, and the elastomer matrix is vulcanized and, accordingly, chemically cross-linked.

The interconnected nanofiber network structure of the semiconducting polymer formed in the elastomer matrix may provide stretchability.

When tensile-deformed due to the vulcanization, the interconnected semiconductor nanofiber network may be maintained.

A mixing ratio of the semiconducting polymer to the elastomer matrix may be from 9:1 to 3:7.

A field-effect mobility of the stretchable semiconductor film having biocompatibility according to the mixing ratio may be maintained in a range of 5.0×10⁻¹ cm²/V·s to 5.5×10⁻¹ cm²/V·s.

The elastomer matrix may be bromo isobutylene isoprene rubber (BIIR).

In accordance with another aspect of the present disclosure, provided is An implantable bioelectronic device, including: a substrate; a gate electrode formed on the substrate; a gate insulating layer formed on the gate electrode; a stretchable semiconductor layer having biocompatibility formed on the gate insulating layer; and a source electrode and drain electrode formed to be spaced apart from each other on the stretchable semiconductor layer, wherein the gate electrode, the source electrode, and the drain electrode are configured as a bilayer electrode composed of gold and silver, the stretchable semiconductor layer having biocompatibility includes: an elastomer matrix having biocompatibility; and a semiconducting polymer of an interconnected semiconductor nanofiber network embedded in the elastomer matrix, wherein the semiconducting polymer of the interconnected semiconductor nanofiber network is formed by phase separation due to a difference in surface energy between the semiconducting polymer and the elastomer matrix during mixing of the semiconducting polymer and the elastomer matrix, and the elastomer matrix is vulcanized and, accordingly, chemically cross-linked.

The bilayer electrode may prevent corrosion in biological environments.

A resistance of the implantable bioelectronic device using the bilayer electrode may be maintained in a range of 0.5Ω to 2Ω in a biological environment.

Field-effect mobility of the implantable bioelectronic device under multiple stretching conditions may be maintained in a range of 1.3×10⁻¹ cm²/V·s to 2.3×10⁻¹ cm²/V·s.

### BRIEF DESCRIPTION OF THE DRAWINGS

The above and other objects, features and other advantages of the present disclosure will be more clearly understood from the following detailed description taken in conjunction with the accompanying drawings, in which:

FIG. 1 illustrates the schematic diagram of a stretchable semiconductor film having biocompatibility according to an embodiment of the present disclosure;

FIG. 2 is a schematic diagram illustrating cross-linking through vulcanization of an elastomer matrix included in the stretchable semiconductor film having biocompatibility according to an embodiment of the present disclosure;

FIG. 3 illustrates the FT-IR spectra of the stretchable semiconductor film having biocompatibility according to an embodiment of the present disclosure before and after vulcanization;

FIG. 4 illustrates the FT-IR spectra of bromo isobutylene isoprene rubber before and after vulcanization;

FIG. 5 illustrates the FT-IR spectra of the stretchable semiconductor film having biocompatibility according to an embodiment of the present disclosure before and after vulcanization, as well as the FT-IR spectrum of the semiconducting polymer;

FIG. 6 illustrates the UV-Vis absorption spectra (left) of the stretchable semiconductor film having biocompatibility according to an embodiment of the present disclosure before and after vulcanization, as well as the field-effect mobility (right) of transistors fabricated from the films before and after vulcanization;

FIG. 7 illustrates the elastic modulus mapping results of the stretchable semiconductor film having biocompatibility according to an embodiment of the present disclosure before and after vulcanization;

FIG. 8 illustrates the schematic diagram of the implantable bioelectronic device according to another embodiment of the present disclosure;

FIG. 9 illustrates the schematic diagram of a fabrication process of the implantable bioelectronic device according to another embodiment of the present disclosure;

FIGS. 10 to 14 illustrate the AFM phase images of Examples 1 to 5;

FIG. 15 is a graph illustrating the field-effect mobility values of the semiconductor films in Examples 1 to 5, according to the mixing ratio of DPPT-TT and BIIR;

FIGS. 16 to 20 illustrate the optical microscope images of Examples 1 to 5 before tensile deformation;

FIGS. 21 to 23 illustrate the optical microscope images of Examples 1 to 3 under 25% tensile strain;

FIGS. 24 and 25 illustrate the optical microscope images of Examples 4 and 5 under 100% tensile strain;

FIG. 26 is a graph illustrating the result of XPS depth profiling performed on Example 4;

FIG. 27 is a set of graphs illustrating the transfer characteristics (left) and output characteristics (right) of a transistor fabricated using Example 4;

FIG. 28 is a graph illustrating the dichroic ratio of Example 4 according to a tensile strain ratio;

FIGS. 29 and 30 illustrate the atomic force microscopy (AFM) and conductive atomic force microscopy (C-AFM) images of Example 4 before and after 50% tensile strain;

FIGS. 31 and 32 illustrate the field-effect mobility of transistors fabricated using Example 4 under tensile deformation;

FIGS. 33 to 35 illustrate the optical microscope images of Comparative Example 1, Comparative Example 2, and Example 7 after immersion in phosphate-buffered saline (PBS) for 72 hours;

FIGS. 36 to 38 are graphs illustrating the changes in resistance of Comparative Example 1, Comparative Example 2, and Example 7 in deionized (DI) water, saline solution, and PBS solution, respectively;

FIG. 39 is a set of graphs illustrating the XPS depth profiling results of the Ag/Au electrode and Example 4;

FIG. 40 is a set of graphs illustrating the transfer characteristics of the transistors of Comparative Example 1, Comparative Example 2, and Example 7, dependent upon source and drain electrode materials;

FIG. 41 is a set of graphs illustrating the resistance measurements of Comparative Example 1, Comparative Example 2, and Example 7 before and after immersion in PBS solution, under tensile deformation ranging from 0% to 50% and in the relaxed state;

FIG. 42 is a set of graphs illustrating the transfer characteristics of Example 7 at drain voltages ranging from −1 V to −60 V;

FIG. 43 is a set of graphs illustrating the field-effect mobility and on/off current ratio of Example 7 measured under tensile deformation from 0% to 50% in vertical and horizontal directions, as well as in a relaxed state;

FIG. 44 illustrates the results of field-effect mobility and on/off current ratio measurements for Example 7 under multiple stretching cycles at a 50% tensile deformation condition;

FIG. 45 is a set of graphs illustrating the change in drain current value after immersion in deionized water, saline solution, and PBS solution for 72 hours;

FIG. 46 illustrates the 5×5 field-effect mobility mapping results of Example 8;

FIG. 47 illustrates the on-current distribution (left) and on/off ratio (right) of 25 devices in Example 8;

FIG. 48 illustrates the current (top) and field-effect mobility (bottom) of Example 8 under a tensile deformation rate ranging from 0% to 50%;

FIG. 49 illustrates the schematic overview of the cell viability assessment;

FIGS. 50 to 55 illustrate the results of the live/dead assay and the CCK-8 assay for BIIR, SEBS, the semiconductor film (blend film) of Example 4, and the logic device;

FIG. 56 illustrates in-vitro wound closure assay images in which human dermal fibroblasts (hDF) were co-cultured with the semiconductor film;

FIG. 57 illustrates graphs quantifying the number of cells in the wound area for each group after cell migration;

FIG. 58 illustrates the results of gene expression analysis related to proliferation, apoptosis, and migration of HDF using qRT-PCR;

FIG. 59 is a set of graphs quantifying the relative gene expression patterns of macrophages (Mφ, Raw264;7 cells) through an antibacterial test for the material groups of BIIR, SEBS, Example 4's semiconductor film (blend film), and the logic device;

FIGS. 60 to 69 illustrate green fluorescence images of genetically modified Escherichia coli (E. coli, top) and Staphylococcus aureus (S. aureus, bottom) for each material group of BIIR, SEBS, Example 4's semiconductor film (blend film), and logic device;

FIGS. 70 and 71 illustrate graphs quantifying the cell viability of genetically modified E. coli (top) and S. aureus (bottom) for the same material groups;

FIG. 72 is a schematic diagram illustrating the experimental method for evaluating the in-vivo biocompatibility of the stretchable semiconductor film having biocompatibility according to the present disclosure, using a subcutaneous implant model in BALB/C mice;

FIG. 73 illustrates a hierarchical clustering heatmap of gene expression in the sham group and the semiconductor film implantation group;

FIG. 74 illustrates the distribution of gene expression levels measured in both groups;

FIG. 75 illustrates the principal component analysis (PCA) scores based on gene expression measurements for the two groups;

FIG. 76 is a graph comparing macrophage phenotype markers between the sham group and the semiconductor film implantation group, based on Western blot analysis;

FIGS. 77 to 79 illustrate the hematoxylin and eosin (H&E) staining results of skin tissues harvested from the implantation sites of the sham group and the semiconductor film implantation group;

FIGS. 80 to 83 illustrate the immunohistochemical staining results for M1 and M2 macrophage markers in the sham group and the semiconductor film implantation group;

FIG. 84 is the schematic diagram of the subcutaneous implantation of a logic circuit fabricated with an implantable transistor;

FIG. 85 illustrates a photograph of an inverter circuit based on CMOS design;

FIG. 86 illustrates the voltage transfer characteristics (VTC) of an inverter under 0% strain (left) and 50% strain (right) before and after implantation in a mouse;

FIG. 87 illustrates the gain of the inverter under the same strain conditions before and after implantation;

FIGS. 88 and 89 illustrate photographs of NOR and NAND circuits based on CMOS design;

FIGS. 90 and 91 illustrate graphs analyzing the input and output characteristics of the NOR and NAND devices under 0% strain and 50% strain, before and after implantation in mice;

FIG. 92 illustrates a tissue image of the logic circuit implantation site; and

FIG. 93 illustrates a graph quantifying the Western blot bands for M1 and M2 macrophage markers and inflammation-related markers.

### DETAILED DESCRIPTION OF THE DISCLOSURE

The present disclosure will now be described more fully with reference to the accompanying drawings and contents disclosed in the drawings. However, the present disclosure should not be construed as limited to the exemplary embodiments described herein.

The terms used in the present specification are used to explain a specific exemplary embodiment and not to limit the present inventive concept. Thus, the expression of singularity in the present specification includes the expression of plurality unless clearly specified otherwise in context. It will be further understood that the terms “comprise” and/or “comprising”, when used in this specification, specify the presence of stated components, steps, operations, and/or elements, but do not preclude the presence or addition of one or more other components, steps, operations, and/or elements thereof.

It should not be understood that arbitrary aspects or designs disclosed in “embodiments”, “examples”, “aspects”, etc. used in the specification are more satisfactory or advantageous than other aspects or designs.

In addition, the expression “or” means “inclusive or” rather than “exclusive or”. That is, unless otherwise mentioned or clearly inferred from context, the expression “x uses a or b” means any one of natural inclusive permutations.

In addition, as used in the description of the disclosure and the appended claims, the singular forms “a”, “an” and “the” are intended to include the plural forms as well, unless context clearly indicates otherwise.

In addition, when an element such as a layer, a film, a region, and a constituent is referred to as being “on” another element, the element can be directly on another element or an intervening element can be present.

Hereinafter, exemplary embodiments of the present disclosure will be described in detail with reference to the accompanying drawings.

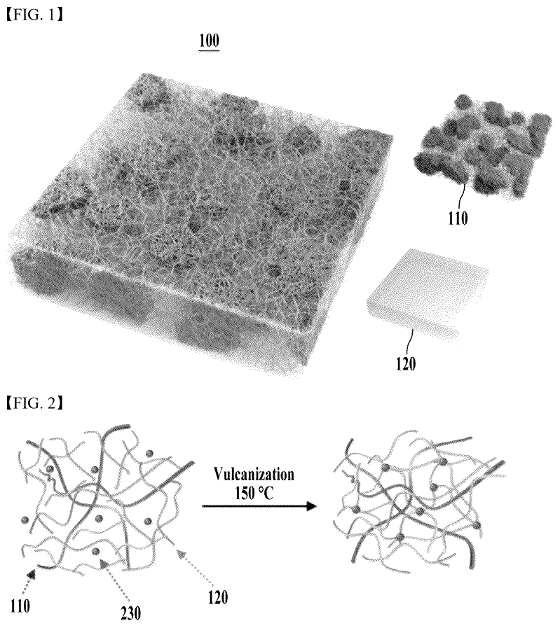

FIG. 1 illustrates the schematic diagram of a stretchable semiconductor film having biocompatibility according to an embodiment of the present disclosure.

Referring to FIG. 1, a stretchable semiconductor film 100 having biocompatibility includes an elastomer matrix 120 having biocompatibility, and a semiconducting polymer 110 that forms an embedded and interconnected semiconductor nanofiber network within the elastomer matrix.

The semiconducting polymer 110 may be any conjugated polymer semiconductor having a surface energy similar to that of the elastomer matrix. As one example of the semiconducting polymer 110, poly [(dithiophene)-alternate-(2,5-bis(2-octyldodecyl)-3,6-bis(thienyl)-diketopyrrolopyrrole)], represented by Formula 1 (hereinafter referred to as DPPT-TT), may be used; however, the configuration of the present disclosure is not limited thereto.

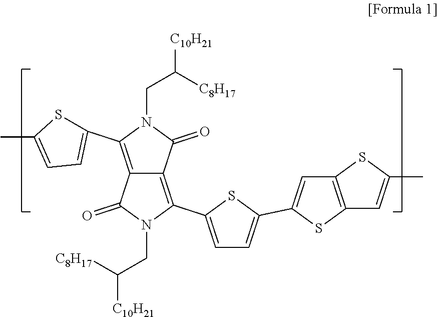

Since a diketopyrrolopyrrole-based polymer used as the semiconducting polymer 110 contains fused thiophene rings, it may exhibit liquid crystallinity, allowing macroscopic alignment in a uniform direction. This alignment enables dense accumulation of π-electron systems between molecules and maximizes intermolecular charge transport via a hopping mechanism between adjacent molecules, thereby exhibiting excellent organic semiconductor properties such as high charge mobility.

The elastomer matrix 120 may include bromo isobutylene isoprene rubber (BIIR), represented by Formula 2 below:

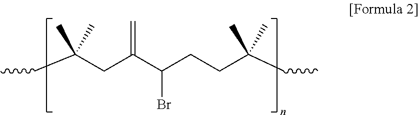

Bromo isobutylene isoprene rubber is a medical-grade elastomer specifically designed for biomedical applications, meeting strict biocompatibility standards established by health authorities such as ISO 10993 and the European Pharmacopoeia (EP). It may exhibit excellent mechanical properties including impact absorption, low permeability, aging resistance, and high physical strength, as well as high chemical resistance and low reactivity with microorganisms.

In the stretchable semiconductor film having biocompatibility according to an embodiment of the present disclosure, the semiconducting polymer 110 is formed in an embedded form within the elastomer matrix 120, and may form an interconnected semiconductor nanofiber network within the elastomer matrix 120.

The interconnected semiconductor nanofiber network of the semiconducting polymer 110 may be formed by phase separation caused by the difference in surface energy between the semiconducting polymer 110 and the elastomer matrix 120 during their mixing.

More specifically, in a stretchable semiconductor film having biocompatibility according to an embodiment, when DPPT-TT is used as the semiconducting polymer 110 and BIIR is used as the elastomer matrix 120, DPPT-TT has a surface energy of 33.4 mJ/m², and BIIR has a surface energy of 36.4 mJ/m². As a result, phase separation between DPPT-TT and BIIR may occur; however, due to the high miscibility between DPPT-TT and BIIR, phase separation in the form of nanofibers may take place.

In the stretchable semiconductor film having biocompatibility according to an embodiment of the present disclosure, stretchability may be imparted by the interconnected nanofiber network of the semiconducting polymer 110 in an embedded form within the elastomer matrix 120.

The elastomer matrix 120 included in an embodiment of the present disclosure is characterized in that it is vulcanized and chemically cross-linked.

The stretchable semiconductor film having biocompatibility according to an embodiment of the present disclosure, which includes the semiconducting polymer 110 and the elastomer matrix 120 may further include a crosslinking agent, a vulcanization initiator and a vulcanization accelerator. These components may connect the chains of the elastomer matrix, thereby improving the mechanical properties of the elastomer matrix 120.

The vulcanization initiator acts as a catalyst or initiator for the crosslinking reaction and may lower the vulcanization temperature. A non-limiting example of a substance that may be used as a vulcanization initiator is stearic acid; however, the configuration of the present disclosure is not limited thereto, and any material commonly used in the relevant technical field may be employed without limitation.

The vulcanization accelerator serves to shorten the vulcanization time, and specifically, dipentamethylenethiuram tetrasulfide (DPTT) may be used; however, the configuration of the present disclosure is not limited thereto.

FIG. 2 is a schematic diagram illustrating cross-linking through vulcanization of an elastomer matrix included in the stretchable semiconductor film having biocompatibility according to an embodiment of the present disclosure.

Referring to FIG. 2, during the formation of the stretchable semiconductor film having biocompatibility according to an embodiment of the present disclosure, chemical cross-linking of the elastomer matrix 120 chains may occur due to the presence of a crosslinking agent 230, which connects the chains to one another.

Hereinafter, the vulcanization crosslinking process of the elastomer matrix will be described using bromo isobutylene isoprene rubber, which is the elastomer matrix 120 used in an embodiment of the present disclosure.

The bromo isobutylene isoprene rubber used as the elastomer matrix 120 exhibits good crosslinking reactivity because the bromine (Br) bonded by heat may be easily dissociated. The vulcanization mechanism consists of three stages: initiation, propagation, and termination.

In the initiation stage where bromine is dissociated at temperatures above 130° C., a radical may be generated on the chain of the bromo isobutylene isoprene rubber by a hydrogen ion (H⁺) from stearic acid, as shown in Formula 3. Alternatively, as shown in Formula 4, sulfur molecules (S₈) used as a crosslinking agent may be decomposed to form sulfur radicals.

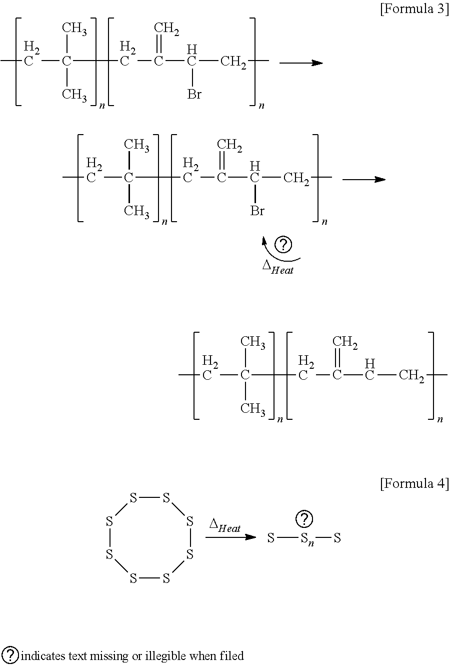

In the propagation stage, as shown in Formula 5, the sulfur radicals generated during the initiation stage may react with the radical formed on the bromo isobutylene isoprene rubber chain or may attack the double bond of the rubber chain to form another reactive site.

In addition, in the propagation stage, dipentamethylenethiuram tetrasulfide (DPTT) may bind with a sulfur radical and be transformed into a molecule (DPTT-Sₓ) having a long sulfur chain between two pentamethylenethiuram groups, and the radicals of bromo isobutylene isoprene rubber generated according to Formula 3 in the initiation stage may react with the sulfur molecules in DPTT-Sₓ, as shown in Formula 6.

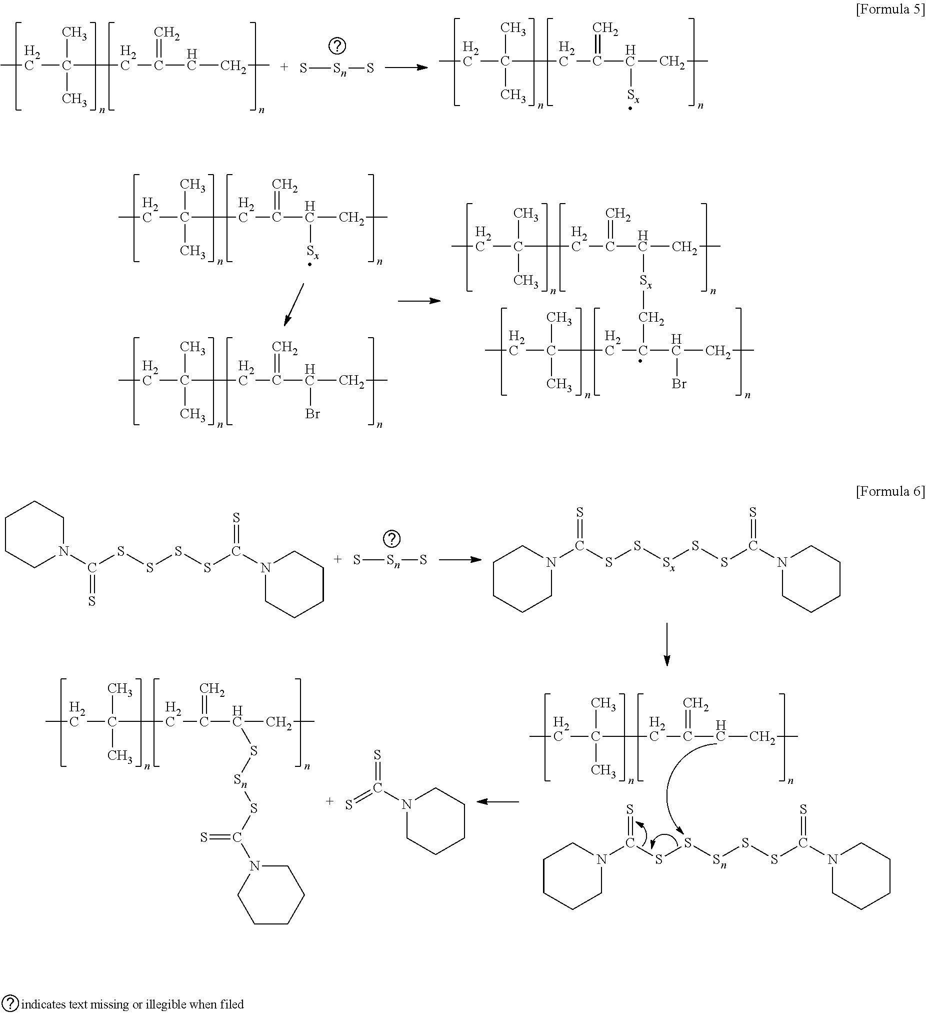

In the termination stage, other radicals of neighboring polymer chains may react with the sulfur atoms of DPTT-Sₓ that are bonded to the polymer chains, as shown in Formula 7. Alternatively, as shown in Formula 8, an unreacted radical may couple with another unreacted radical, or as shown in Formula 9, the unreacted radical may bind to pentamethylenethiuram. When all radicals have reacted through the above processes, the vulcanization reaction is completed.

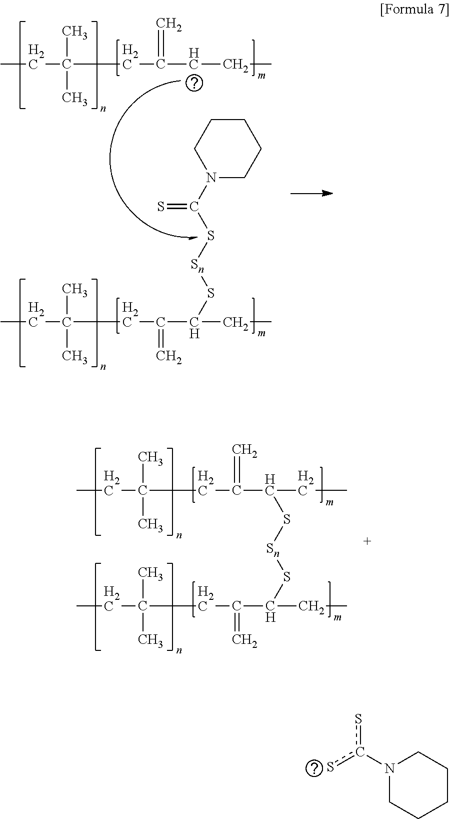

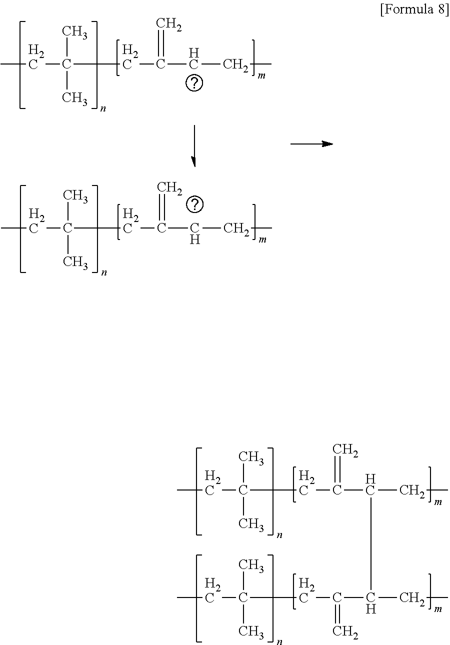

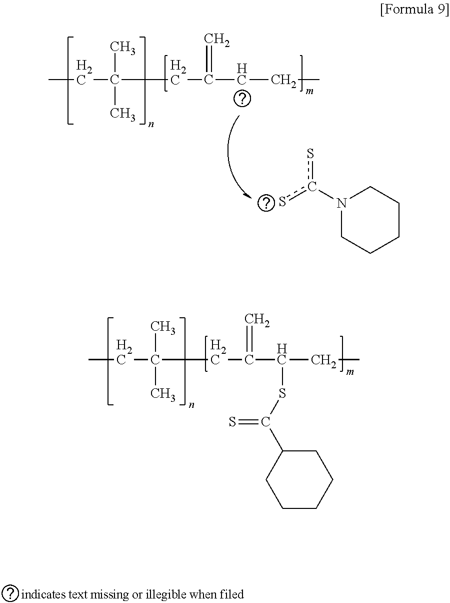

As shown in Formulas 1 to 9, the crosslinking of the elastomer matrix 120 through vulcanization proceeds only through the connection between the chains of the elastomer matrix 120, while the conjugated bonding structure of the semiconducting polymer 110 is preserved. Therefore, the excellent electrical properties resulting from the conjugated bonding structure of the semiconducting polymer 110 may be maintained even after vulcanization.

In addition, after vulcanization, the mechanical properties of an elastomer matrix 220, such as modulus and elasticity, are improved, and the nanofiber network of the semiconducting polymer embedded within the elastomer matrix may be preserved even under tensile strain of up to 100%.

Hereinafter, the structures and characteristics of the elastomer matrix, semiconducting polymer, and a stretchable semiconductor film having biocompatibility before and after vulcanization will be described in detail.

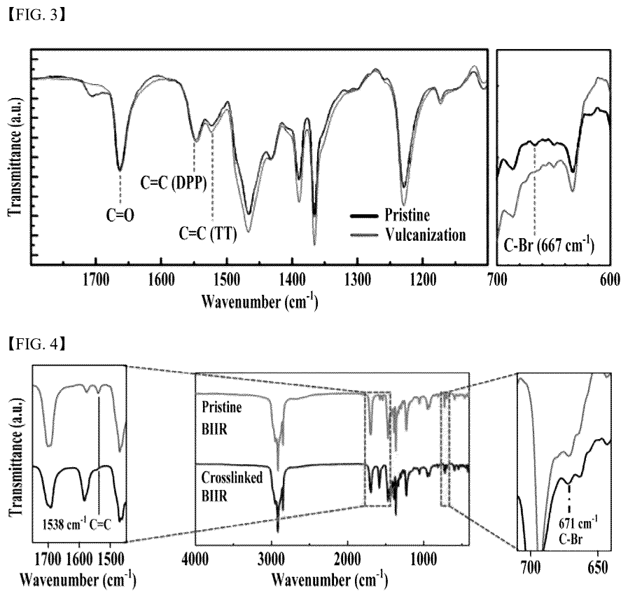

FIG. 3 illustrates the FT-IR spectra of the stretchable semiconductor film having biocompatibility according to an embodiment of the present disclosure before and after vulcanization, and FIG. 4 illustrates the FT-IR spectra of bromo isobutylene isoprene rubber before and after vulcanization.

Referring to FIGS. 3 and 4, it can be confirmed that the initiation and propagation stages of the vulcanization reaction have proceeded, as evidenced by a significant decrease in the carbon-bromine (C—Br) peak at 667 cm⁻¹ and the carbon-carbon double bond (C═C) peak at 1538 cm⁻¹.

In addition, FIG. 5 illustrates the FT-IR spectra of the stretchable semiconductor film having biocompatibility according to an embodiment of the present disclosure before and after vulcanization, as well as the FT-IR spectrum of the semiconducting polymer.

Referring to FIG. 5, the carbon-carbon double bond (C═C) peak shows no significant difference before and after vulcanization, indicating that the conjugated bonding structure of the semiconducting polymer is preserved even after vulcanization.

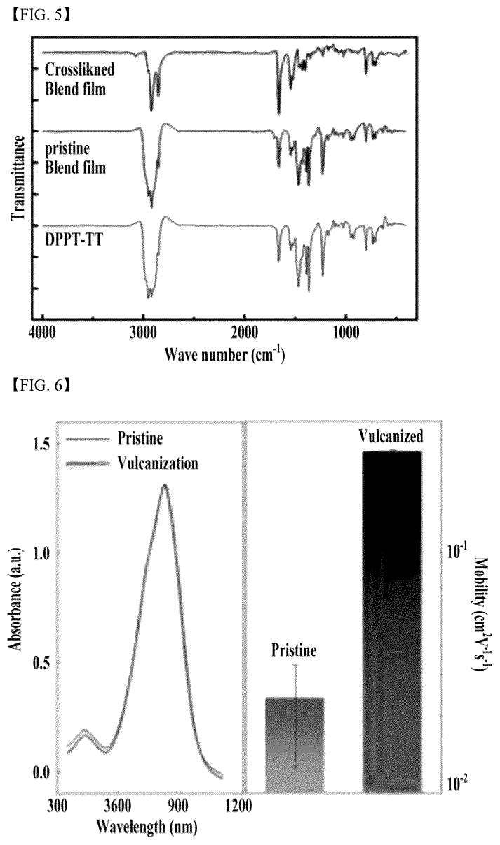

FIG. 6 illustrates the UV-Vis absorption spectra (left) of the stretchable semiconductor film having biocompatibility according to an embodiment of the present disclosure before and after vulcanization, as well as the field-effect mobility (right) of transistors fabricated from the films before and after vulcanization.

Referring to FIG. 6, it can be seen that there is no significant difference between the UV-Vis absorption spectrum after vulcanization and that before vulcanization of the stretchable semiconductor film having biocompatibility according to an embodiment of the present disclosure.

This result suggests that the vulcanization process selectively crosslinks the elastomer matrix chains without damaging the conjugated molecular structure of the semiconducting polymer.

In addition, referring to FIG. 6, it can be observed that the field-effect mobility value increases significantly after the vulcanization of the elastomer matrix. This may be attributed to the uniform distribution of sulfur(S) atoms throughout the semiconductor film after vulcanization, which enables charge transport and mobility in both the vertical and horizontal directions of the film. Furthermore, the heat generated during the vulcanization process may optimize the morphology of the semiconductor film, thereby enhancing the field-effect mobility value.

FIG. 7 illustrates the elastic modulus mapping results of the stretchable semiconductor film having biocompatibility according to an embodiment of the present disclosure before and after vulcanization.

Referring to FIG. 7, it can be seen that the Derjaguin-Muller-Toporov (DMT) modulus value increased after vulcanization, and the stretchable semiconductor film having biocompatibility of the present disclosure retains its nanostructure even after the modulus is enhanced through vulcanization.

In the stretchable semiconductor film having biocompatibility according to an embodiment of the present disclosure, the mixing ratio of the semiconducting polymer to the elastomer matrix may range from 9:1 to 3:7. Since the elastomer matrix is an insulating material, increasing its ratio generally leads to a decrease in electrical conductivity.

However, in the stretchable semiconductor film having biocompatibility according to an embodiment of the present disclosure, the semiconducting polymer forms an interconnected nanofiber network in an embedded form within the elastomer matrix, and therefore, even when the elastomer matrix is blended at a ratio of up to 70%, the field-effect mobility value may be maintained.

More specifically, the field-effect mobility value of a transistor fabricated using the stretchable semiconductor film having biocompatibility according to an embodiment of the present disclosure may be maintained in the range of 5.0×10⁻¹ cm²/V·s to 5.5×10⁻¹ cm²/V·s.

Because the stretchable semiconductor film having biocompatibility according to an embodiment of the present disclosure forms an interconnected nanofiber network of the semiconducting polymer in an embedded state within the elastomer matrix, it may maintain high conductivity even when the proportion of semiconducting polymer is reduced and the mixing ratio of the insulating elastomer matrix is increased.

The ability to maintain the field-effect mobility value even when the elastomer matrix is blended up to 70% allows for a reduction in the usage of the high-cost semiconducting polymer, thereby enabling a cost-effective manufacturing process for the biocompatible semiconductor film according to one embodiment of the present disclosure.

In addition, the stretchable semiconductor film having biocompatibility according to an embodiment of the present disclosure may prevent mechanical damage during tensile deformation as the ratio of elastomer matrix increases. More preferably, when the mixing ratio of semiconducting polymer to elastomer matrix is 3:7, tensile deformation up to 100% may be sustained without mechanical cracking.

Moreover, the interconnected nanofiber network of the semiconducting polymer possesses high stretchability, allowing for the field-effect mobility to remain high even under 100% tensile strain.

Hereinafter, a bio-implantable electronic device including the stretchable semiconductor film having biocompatibility according to an embodiment of the present disclosure will be described in detail. The bio-implantable electronic device according to another embodiment of the present disclosure may be a transistor or a logic circuit, but is not limited thereto.

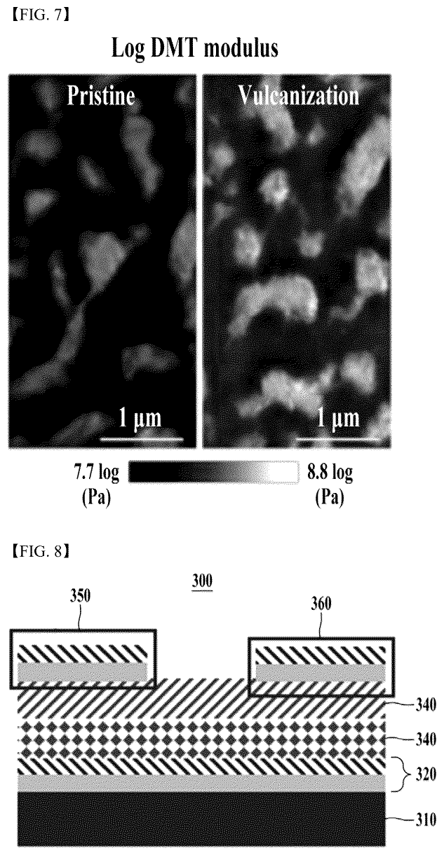

FIG. 8 illustrates the schematic diagram of the implantable bioelectronic device according to another embodiment of the present disclosure.

Referring to FIG. 8, an implantable bioelectronic device 300 may include a substrate 310, a gate electrode 320 formed on the substrate, a gate insulating layer 330 formed on the gate electrode, a biocompatible and stretchable semiconductor layer 340 formed on the gate insulating layer, and source electrode 350 and drain electrode 360 formed on the stretchable semiconductor layer 340 and spaced apart from each other, and the gate electrode 320, the source electrode 350 and the drain electrode 360 may be bilayer electrode composed of gold and silver.

The stretchable semiconductor layer 340 having biocompatibility, which is included in the implantable bioelectronic device 300, may include a biocompatible elastomer matrix; and a semiconducting polymer that forms an interconnected semiconductor nanofiber network embedded within the elastomer matrix.

The semiconducting polymer forming the interconnected semiconductor nanofiber network included in the stretchable semiconductor layer 340 having biocompatibility is formed by phase separation resulting from the difference in surface energy between the semiconducting polymer and the elastomer matrix during the formation of the stretchable semiconductor layer 340. The elastomer matrix is characterized in that it undergoes vulcanization and becomes chemically cross-linked.

FIG. 9 illustrates the schematic diagram of a fabrication process of the implantable bioelectronic device according to another embodiment of the present disclosure.

Referring to FIG. 9, the implantable bioelectronic device may be fabricated by forming a stretchable semiconductor layer having biocompatibility on an OTS-treated SiO₂/Si wafer, then forming a dielectric layer on a substrate having bilayer electrodes composed of gold (Au) and silver (Ag) via physical vapor deposition (PVD). The stretchable semiconductor layer is then transferred onto the dielectric-layered substrate, followed by the formation of source and drain electrodes using a thermal evaporator.

Generally, silver electrodes are widely used in electronic devices due to their high conductivity. However, they may suffer from corrosion in biological environments, and their intrinsic rigidity may lead to mechanical mismatches with tissues, potentially causing tissue damage and inflammation. Thus, the development of electrodes with both biocompatibility and stretchability is necessary.

The implantable bioelectronic device according to another embodiment of the present disclosure enables the fabrication of biocompatible and stretchable electrodes by continuously depositing silver (for high conductivity) and gold (for high corrosion resistance in biological environments) onto the biocompatible and stretchable semiconductor layer using a vapor deposition method.

In addition, the implantable bioelectronic device according to another embodiment of the present disclosure may exhibit high corrosion resistance in in-vivo environments and maintain low electrical resistance even under physiological conditions. More specifically, the resistance of the implantable bioelectronic device including the bilayer electrode according to another embodiment of the present disclosure in biological environments may range from 0.5Ω to 2Ω.

In addition, the implantable bioelectronic device according to another embodiment of the present disclosure may maintain a field-effect mobility value in the range of 1.3×10⁻¹ cm²/V·s to 2.3×10⁻¹ cm²/V·s under multi-stretching conditions.

More specifically, the implantable semiconductor film according to another embodiment of the present disclosure may retain a mobility value of 2.3×10⁻¹ cm²/V·s to 3.9×10⁻¹ cm²/V·s, which is equivalent to the pre-stretching field-effect mobility value, even after being stretched up to 1000 cycles under 100% strain, without mechanical damage or performance degradation. Furthermore, the implantable bioelectronic device that includes the implantable semiconductor film may maintain a field-effect mobility of at least 1.0×10⁻¹ cm²/V·s and an on/off current ratio under tensile strain up to 50%, regardless of the stretching direction.

In addition, the implantable bioelectronic device according to another embodiment of the present disclosure exhibits a low threshold voltage and an on/off ratio of 10⁴ or more, enabling ideal signal transmission and output, and can operate even under low operating voltages suitable for in vivo use.

Hereinafter, the present disclosure will be described in more detail through embodiments. These embodiments are provided to illustrate the present disclosure in more detail and are not intended to limit the scope of the present disclosure.

### A. Materials

Gold (Au, 99.99%, 3 mm granules) and silver (Ag, 99.99%, 3-5 mm granules) were purchased from SYSCIENCE (Seoul, Republic of Korea), and poly [(dithiophene)-alternate-(2,5-bis(2-octyldodecyl)-3,6-bis(thienyl)-diketopyrrolopyrrole)] (DPPT-TT) was purchased from Derthon (Shenzhen, China).

Bromo isobutylene isoprene rubber (BIIR) was provided by Samsung Medical Rubber, and SEBS H1062 and SEBS H1052 were provided by Asahi KASEI. H1062 (S/EB weight ratio, 18/82) and H1052 (S/EB weight ratio, 20/80) were used as an elastic substrate and a dielectric layer, respectively.

Anhydrous toluene (99.8%), anhydrous chlorobenzene (99.8%), anhydrous chloroform (99.8%), sulfur, dipentamethylenethiuram tetrasulfide, trichloro(octadecyl) silane (OTS), and stearic acid were purchased from Sigma Aldrich (St. Louis, Missouri, MO, USA).

Poly(dimethylsiloxane) (PDMS, Sylgard 184) and a curing agent were purchased from Dow Corning (Midland, MI, USA), and PDMS was cured overnight at 55° C. in an 11:1 (base:curing agent, w/w) ratio for stamp fabrication. Silicon wafers (SiO₂/Si, 1-30Ω, 300 nm SiO₂) were purchased from iTASCO. All chemicals were used as received without further purification.

### B. Property Evaluation

To stabilize devices, all devices were aged overnight in an automatic dry desiccator (relative humidity: 24%, temperature: 27° C.). Electrical properties were measured under ambient conditions using a probe station connected to a KEITHLEY 4200, and a vacuum probe station equipped with a Peltier device for temperature control connected to a KEITHLEY 2636B.

Dielectric capacitance was measured using a probe station connected to an LCR meter (Keysight 4274A). UV-Vis-NIR spectroscopy was performed using a Jasco V-770 spectrophotometer.

Surface morphology was observed under ambient conditions using atomic force microscopy (AFM; Bruker MultiMode 8-HR), and optical microscope (OM) images were obtained using a Leica 2DM4 M.

Film thickness was measured using an ellipsometer (WONWOO STRC-2000). To obtain stretched OM and AFM images, a semiconductor was transferred using the PDMS stamp method.

### Fabrication of Stretchable Semiconductor Film Having Biocompatibility and Property Evaluation Thereof

### 1. Preparation of DPPT-TT/BIIR Blend Solution

### Preparation Example 1

Poly [(dithiophene)-alternate-(2,5-bis(2-octyldodecyl)-3,6-bis(thienyl)-diketopyrrolopyrrole)] (hereinafter referred to as DPPT-TT) and bromo isobutylene isoprene rubber (hereinafter referred to as BIIR) were dissolved in anhydrous chlorobenzene at a concentration of 0.8 wt % according to the ratios shown in Table 1, and the solution was stirred at 120° C. for 4 hours.

TABLE 1

| | Preparation | Preparation | Preparation | Preparation | Preparation |
|---|---|---|---|---|---|
| | Example 1-1 | Example 1-2 | Example 1-3 | Example 1-4 | Example 1-5 |
| DPPT-TT | 9 | 7 | 5 | 3 | 1 |
| BIIR | 1 | 3 | 5 | 7 | 9 |

### 2. Fabrication of DPPT-TT/BIIR Blend Film (Film without Vulcanization Process)

### Preparation Example 2

The solution mixed at the ratio of Preparation Example 1-1 was filtered using a PTFE-D filter (0.2 μm), spin-coated on an OTS-treated SiO₂/Si wafer at 800 rpm for 1 minute, and then annealed at 150° C. for 1 hour to prepare Preparation Example 2. The entire process was carried out in a nitrogen (N₂) atmosphere glove box with H₂O<0.01 ppm and 02<0.01 ppm.

### Preparation Example 3

Preparation Example 3 was fabricated in the same manner as Preparation Example 2, except that the solution mixed at the ratio of Preparation Example 1-2 was used instead of that of Preparation Example 1-1.

### Preparation Example 4

Preparation Example 4 was fabricated in the same manner as Preparation Example 2, except that the solution mixed at the ratio of Preparation Example 1-3 was used instead of that of Preparation Example 1-1.

### Preparation Example 5

Preparation Example 5 was fabricated in the same manner as Preparation Example 2, except that the solution mixed at the ratio of Preparation Example 1-4 was used instead of that of Preparation Example 1-1.

### Preparation Example 6

Preparation Example 6 was fabricated in the same manner as Preparation Example 2, except that the solution mixed at the ratio of Preparation Example 1-5 was used instead of that of Preparation Example 1-1.

### 3. Fabrication of Vulcanized DPPT-TT/BIIR Semiconductor Film (Fabrication of Stretchable Semiconductor Film Having Biocompatibility)

### Example 1

A semiconductor solution was prepared by mixing Preparation Example 1-1 with sulfur (Sa), dipentamethylenethiuram tetrasulfide (DPTT), and stearic acid. BIIR, sulfur, DPTT, and stearic acid were added at a ratio of 100:1:1:5.

The prepared semiconductor solution was filtered using a PTFE-D filter (0.2 μm), spin-coated on an OTS-treated SiO₂/Si wafer at 800 rpm for 1 minute, and then annealed at 150° C. for 1 hour to fabricate Example 1. The entire process was carried out in a glove box under nitrogen (N₂) atmosphere with H₂O<0.01 ppm and 02<0.01 ppm.

### Example 2

Example 2 was prepared in the same manner as Example 1, except that Preparation Example 1-2 was used instead of Preparation Example 1-1 in Example 1.

### Example 3

Example 3 was prepared in the same manner as Example 1, except that Preparation Example 1-3 was used instead of Preparation Example 1-1 in Example 1.

### Example 4

Example 4 was prepared in the same manner as Example 1, except that Preparation Example 1-4 was used instead of Preparation Example 1-1 in Example 1.

### Example 5

Example 5 was prepared in the same manner as Example 1, except that Preparation Example 1-5 was used instead of Preparation Example 1-1 in Example 1.

### [Example 6] Fabrication of Organic Thin-Film Transistor on Rigid Substrate

A semiconductor solution was prepared by mixing Preparation Example 1-4 with sulfur (S₈), dipentamethylenethiuram tetrasulfide (DPTT), and stearic acid. BIIR, sulfur, DPTT, and stearic acid were added at a ratio of 100:1:1:5.

The prepared semiconductor solution was filtered using a PTFE-D filter (0.2 μm), spin-coated on an OTS-treated SiO₂/Si wafer at 800 rpm for 1 minute, and then annealed at 150° C. for 1 hour to fabricate a semiconductor film. The entire process was carried out in a glove box under nitrogen (N₂) atmosphere with H₂O<0.01 ppm and O₂<0.01 ppm.

To prepare a dielectric solution, 60 mg/mL of SEBS H1052 was dissolved in toluene at 60° C. for 4 hours. The solution was spin-coated onto indium tin oxide (ITO) glass substrates (sheet resistance: 20 ohm/square) at 1000 rpm for 1 minute to obtain a SEBS/ITO glass substrate.

The semiconductor film was then transferred from the OTS-treated wafer onto the SEBS/ITO glass substrate using a polydimethylsiloxane (PDMS) stamp.

Source/drain electrodes composed of silver and gold were deposited using a thermal evaporator at a rate of 0.2 nm/s, with the channel length and width set to 1000 μm and 150 μm, respectively.

### [Property Evaluation 1] Morphological Analysis of Stretchable Semiconductor Films Having Biocompatibility According to Mixing Ratio of DPPT-TT and BIIR

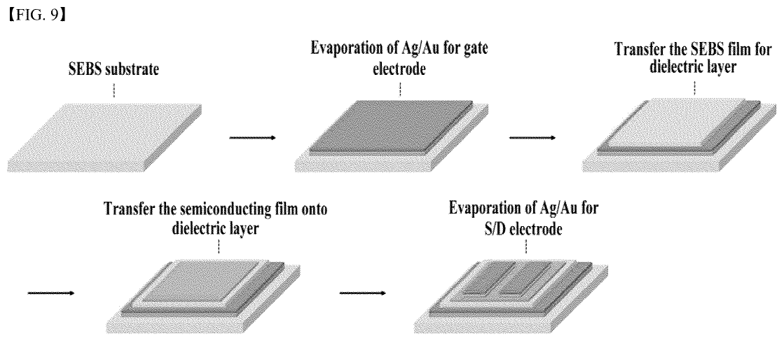

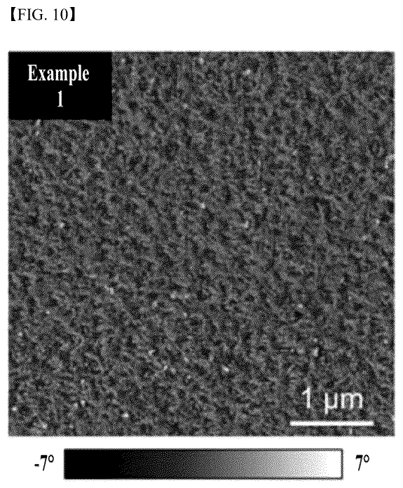

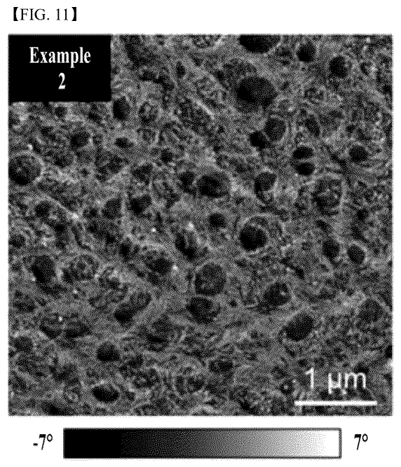

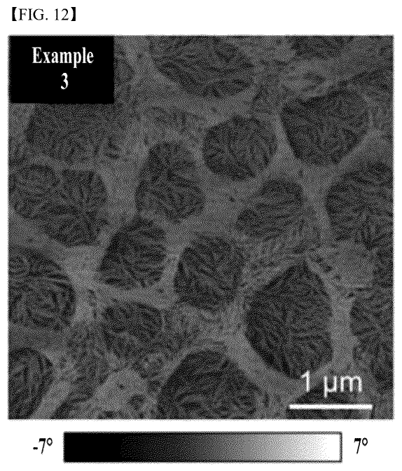

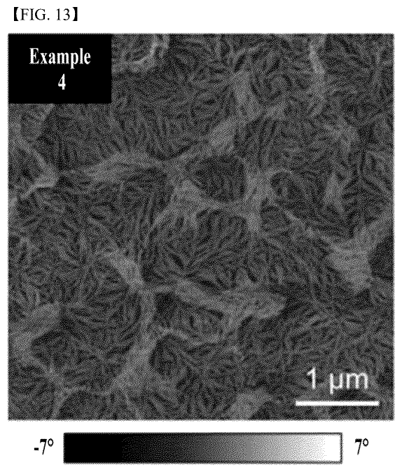

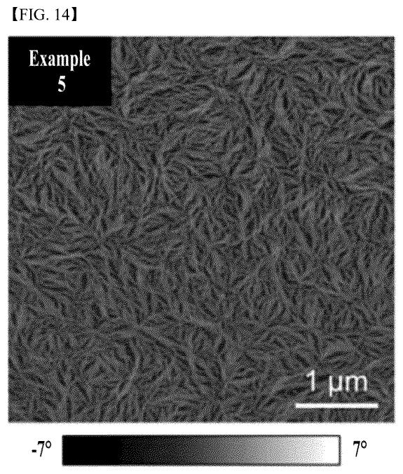

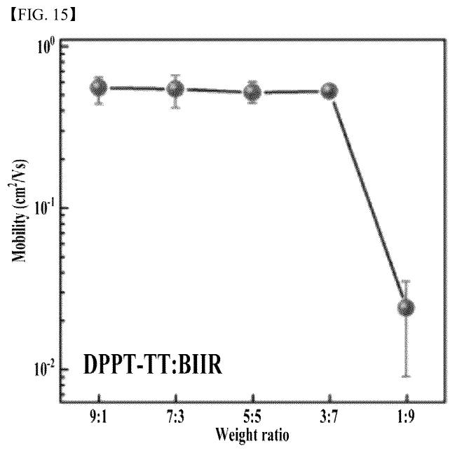

FIGS. 10 to 14 illustrate the AFM phase images of Examples 1 to 5, respectively. More specifically, FIG. 10 illustrates the AFM phase images of Example 1, FIG. 11 illustrates the AFM phase images of Example 2, FIG. 12 illustrates the AFM phase images of Example 3, FIG. 13 illustrates the AFM phase images of Example 4, and FIG. 14 illustrates the AFM phase images of Example 5.

Referring to FIGS. 10 to 14, the semiconductor films of Examples 1 to 5 exhibit nanoscale phase images in which the semiconducting polymer is embedded within the elastomer matrix. As the mixing ratio of the elastomer matrix increases, it can be observed that the semiconducting polymer forms an embedded nanofiber structure within the elastomer matrix.

FIG. 15 is a graph illustrating the field-effect mobility values of the semiconductor films in Examples 1 to 5, according to the mixing ratio of DPPT-TT and BIIR.

Referring to FIG. 15, the semiconductor films in Examples 1 to 4, where the mixing ratio of DPPT-TT to BIIR ranges from 9:1 to 3:7, maintain field-effect mobility values similar to that of Example 1, in which the highest amount of the semiconducting polymer DPPT-TT was used. This indicates that even when up to 70% of BIIR, an insulating polymer, is used, the percolation pathways of the semiconductor are still preserved.

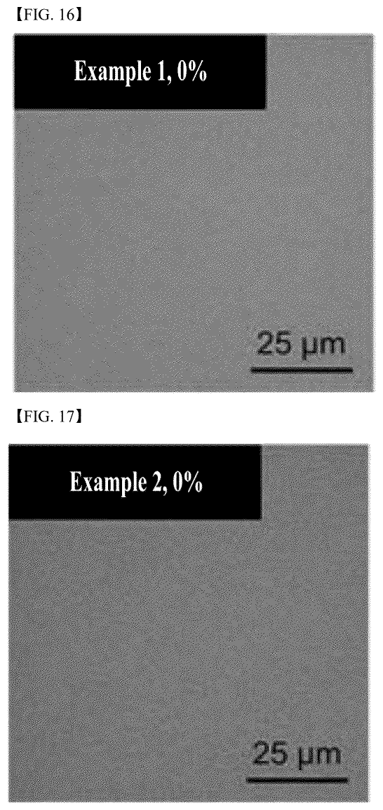

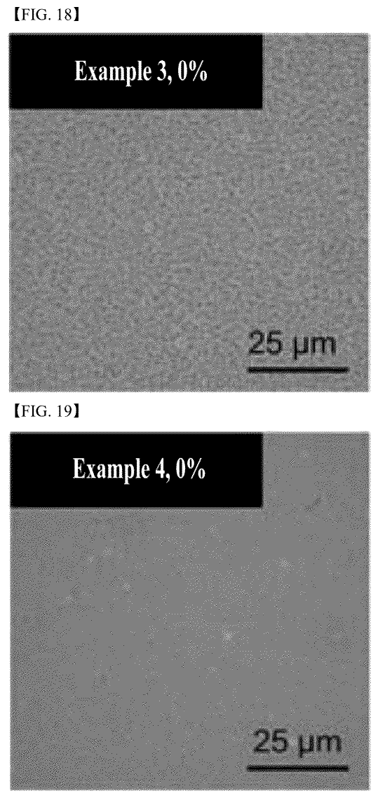

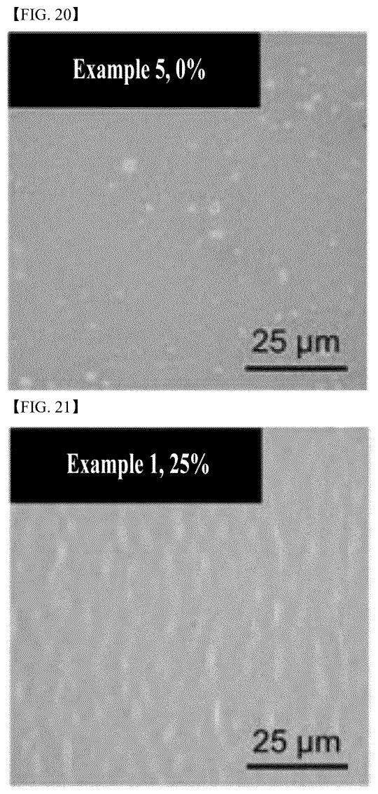

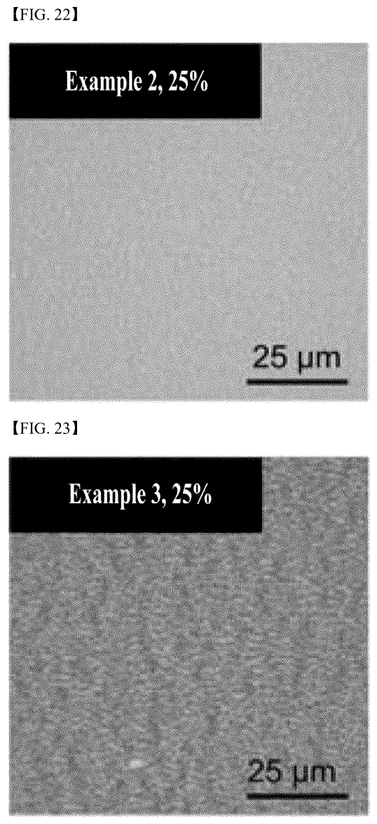

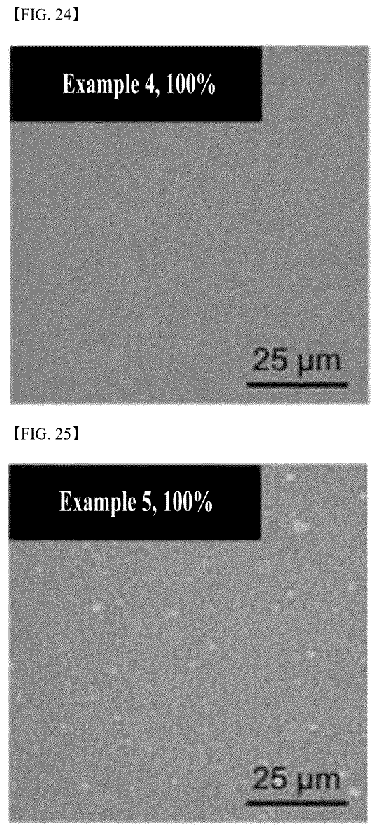

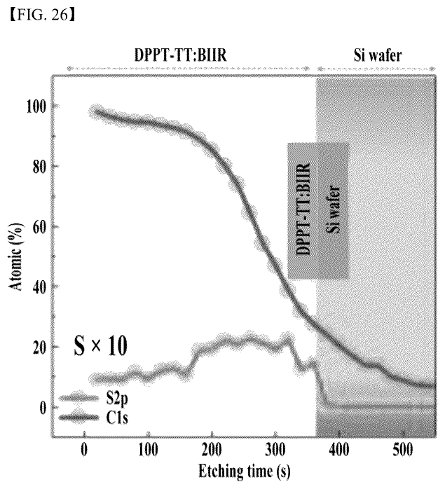

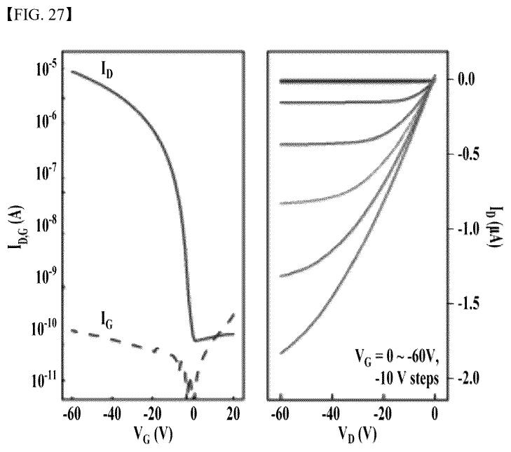

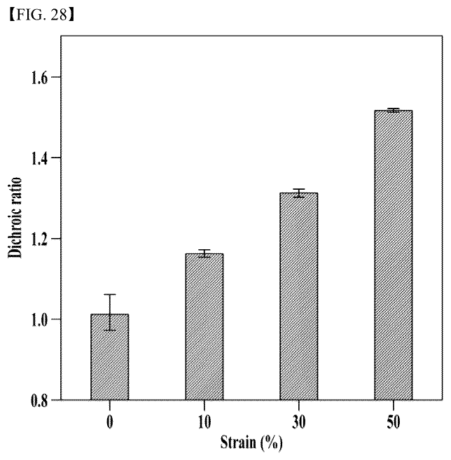

FIGS. 16 to 20 illustrate the optical microscope images of Examples 1 to 5 before tensile deformation, FIGS. 21 to 23 illustrate the optical microscope images of Examples 1 to 3 under 25% tensile strain, and FIGS. 24 and 25 illustrate the optical microscope images of Examples 4 and 5 under 100% tensile strain.

Referring to FIGS. 16 to 25, it was observed that the semiconductor films of Examples 1 to 3, which contain a lower amount of BIIR, exhibited cracks under 25% tensile strain, whereas Example 4, which contains 70% BIIR, showed no mechanical cracking even under 100% tensile strain.

Referring to FIGS. 16 to 25, it can be seen that a higher BIIR content is required to achieve the inherent stretchability of the stretchable semiconductor film having biocompatibility according to the present disclosure. In addition, the optimal mixing ratio of DPPT-TT to BIIR for maintaining the field-effect mobility of the semiconductor nanofiber network, the strain threshold for crack initiation, and overall film integrity is determined to be 3:7.

### [Property Evaluation 2] Property Investigation of Stretchable Semiconductor Film Having Biocompatibility

### 1. X-Ray Photoelectron Spectroscopy (XPS) Profiling of Stretchable Semiconductor Film Having Biocompatibility

FIG. 26 is a graph illustrating the result of XPS depth profiling performed on Example 4.

Referring to FIG. 26, it can be seen that after vulcanization, sulfur(S) atoms are uniformly distributed throughout the semiconductor film, facilitating effective charge injection and transport in both the vertical and horizontal directions.

FIG. 27 is a set of graphs illustrating the transfer characteristics (left) and output characteristics (right) of a transistor fabricated using Example 4.

Referring to FIG. 27, it can be seen that the transistor fabricated using Example 4 exhibits high mobility and a high on/off ratio, indicating that the vulcanization process also functions as a thermal annealing process. This dual function both cross-links the BIIR elastomer and optimizes the morphology of the semiconducting polymer film, resulting in ideal transfer and output characteristics.

FIG. 28 is a graph illustrating the dichroic ratio of Example 4 according to a tensile strain ratio. In this case, the tensile deformation was applied as uniaxial strain.

Referring to FIG. 28, it can be seen that the dichroic ratio increases linearly as a function of the applied strain (ranging from 0% to 50%), which indicates that the DPPT-TT semiconductor nanofibers are aligned along the direction of deformation without mechanical cracking.

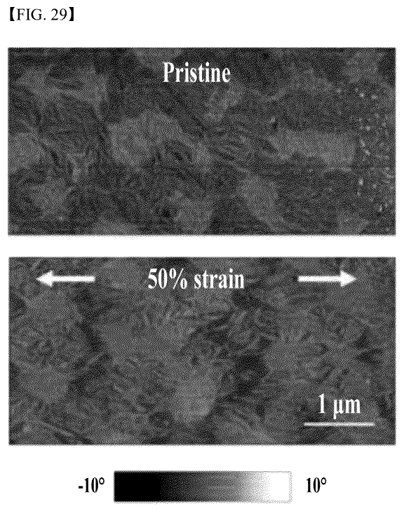

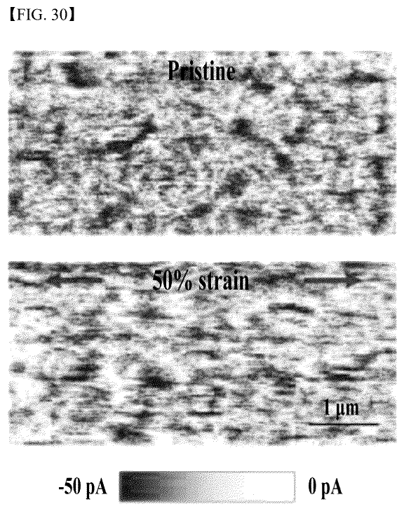

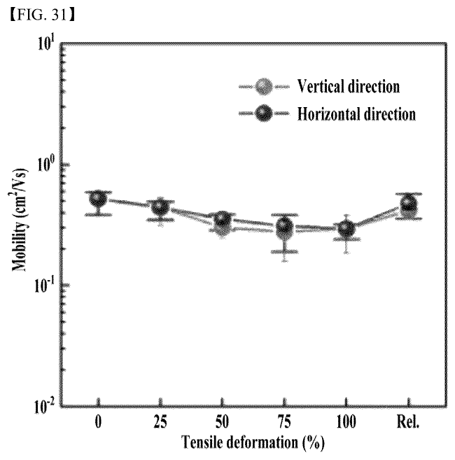

FIGS. 29 and 30 illustrate the atomic force microscopy (AFM) and conductive atomic force microscopy (C-AFM) images of Example 4 before and after 50% tensile strain. More specifically, FIG. 29 presents the AFM observation results, and FIG. 30 presents the C-AFM observation results.

Referring to FIGS. 29 and 30, it can be seen that both the surface morphology of Example 4 and the magnitude of current flow within the film remain similar before and after tensile deformation.

The fact that the internal current of Example 4 remains unchanged after tensile deformation indicates that the conductive pathways of Example 4 are preserved in the same manner before and after deformation.

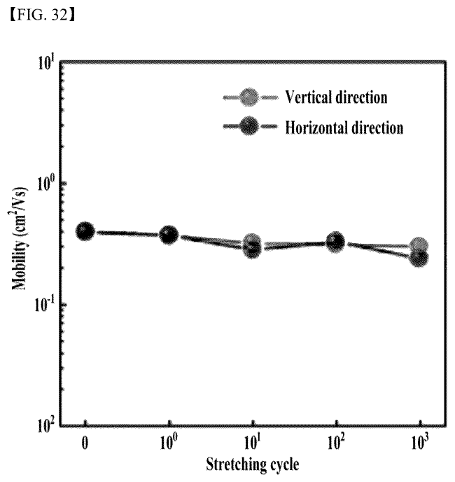

FIGS. 31 and 32 illustrate the field-effect mobility of transistors fabricated using Example 4 under tensile deformation. More specifically, FIG. 31 illustrates the field-effect mobility of the transistor fabricated from Example 4 under tensile deformation ranging from 0% to 100%, as well as in the relaxed state, while FIG. 32 illustrates the field-effect mobility of the transistor fabricated from Example 4 under repeated cycles of 100% tensile deformation.

As shown in FIGS. 31 and 32, even under tensile deformation up to 100%, the variation in field-effect mobility is not significant. Moreover, after 1000 cycles of 100% tensile deformation, the field-effect mobility of Example 4 remains stable, indicating excellent mechanical durability.

Therefore, the stretchable semiconductor film having biocompatibility according to the present disclosure exhibits very high stretchability, and it can be concluded that the nanostructure of the semiconducting polymer DPPT-TT is preserved even under mechanical deformation.

### Fabrication and Characterization Evaluation of Electronic Devices Incorporating Stretchable Semiconductor Film Having Biocompatibility

### 1. Fabrication and Characterization Evaluation of Stretchable Transistors Incorporating Stretchable Semiconductor Film Having Biocompatibility

### [Example 7] Fabrication of Stretchable Transistor

An elastic substrate for the transistor was prepared by casting SEBS H1062 (100 mg/mL in toluene) onto a glass slide and drying it overnight. On the fabricated SEBS substrate, an Au (30 nm)/Ag gate electrode (30 nm) was deposited onto the semiconductor film by thermal evaporation at a rate of 0.2 nm/s under high vacuum (below 5.0×10⁶ torr). The channel length and width were 1000 μm and 150 μm, respectively.

### Comparative Example 1

Comparative Example 1 was prepared in the same manner as Example 6, except that a silver (Ag) electrode was used instead of the silver and gold source/drain electrode in Example 7.

### Comparative Example 2

Comparative Example 2 was prepared in the same manner as Example 6, except that a gold (Au) electrode was used instead of the silver and gold source/drain electrode in Example 7.

### [Property Evaluation 1] Evaluation of Biocompatibility, Stretchability and Conductivity of Transistors Having Silver/Gold Bilayer Electrode

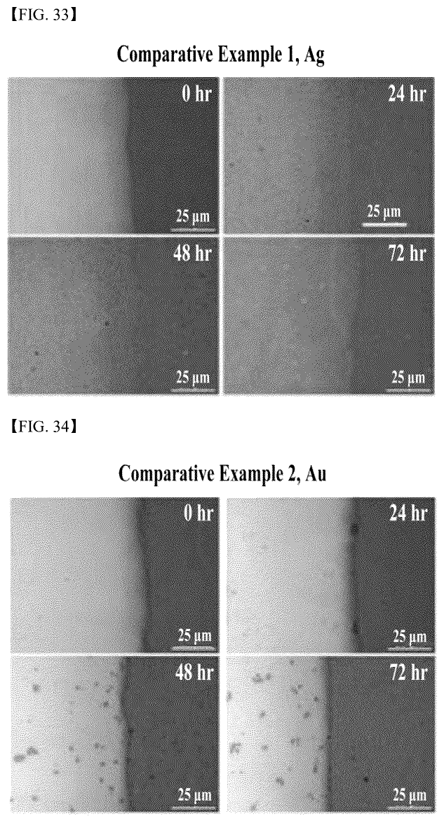

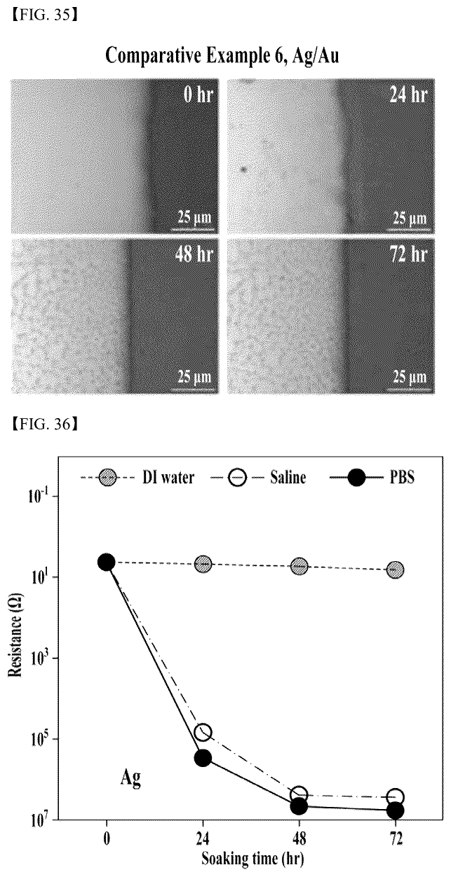

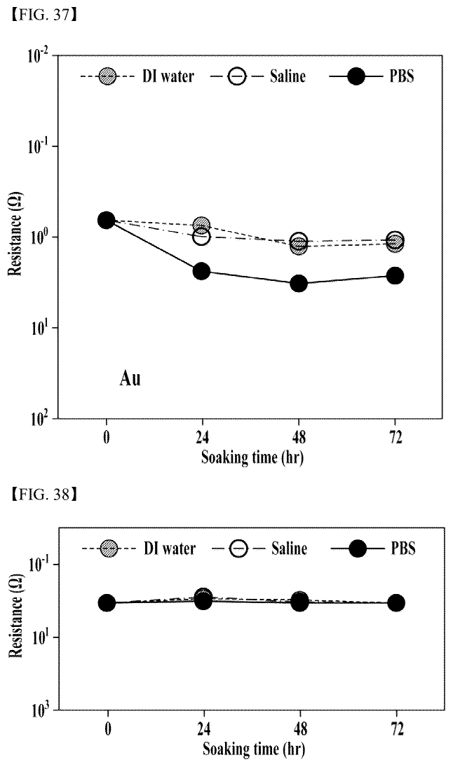

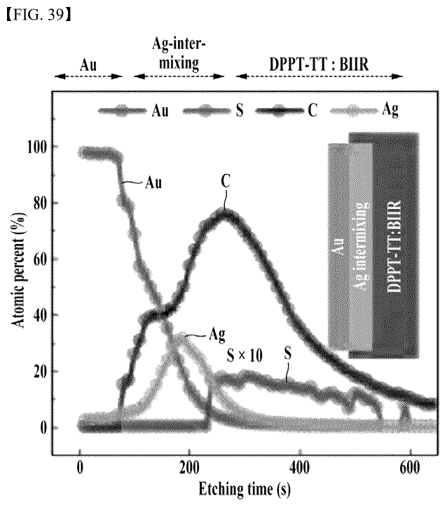

FIGS. 33 to 35 illustrate the optical microscope images of Comparative Example 1, Comparative Example 2, and Example 7 after immersion in phosphate-buffered saline (PBS) for 72 hours. More specifically, FIG. 33 illustrates the observation result for Comparative Example 1, FIG. 34 for Comparative Example 2, and FIG. 35 for Example 7.

As shown in FIGS. 33 to 35, while Comparative Example 1 where the silver electrode was used begins to dissolve in PBS solution after 24 hours, Comparative Example 2 where the gold electrode was used and Example 7 where the silver/gold bilayer electrode was used remain undissolved even after 72 hours in PBS.

The results shown in FIGS. 33 to 35 indicate that gold (Au) can provide strong protection against corrosion caused by biological fluids, thereby enhancing the durability of electrodes in biological environments.

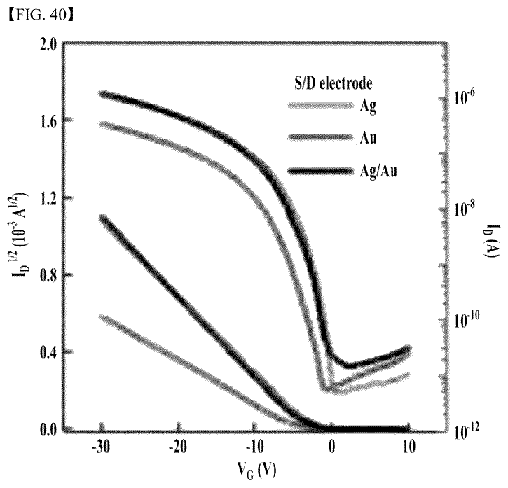

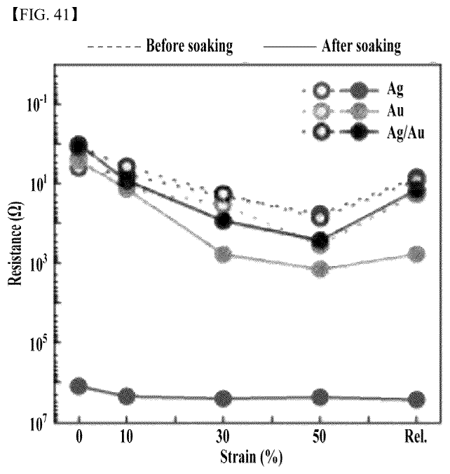

FIGS. 36 to 38 are graphs illustrating the changes in resistance of Comparative Example 1, Comparative Example 2, and Example 7 in deionized (DI) water, saline solution, and PBS solution, respectively.

Referring to FIGS. 36 to 38, it can be seen that Comparative Example 1, which uses a silver electrode, exhibits a significant increase in resistance over time in all immersion conditions except for DI water.

Comparative Example 2, which uses a gold electrode, tends to show a decrease in resistance as the soaking time increases; however, the magnitude of this change is smaller compared to the rate of resistance reduction observed in Comparative Example 1 that uses a silver electrode.

In the case of Example 7 which uses a silver/gold bilayer electrode, the resistance remains constant over time, regardless of the type of immersion solution, even as the soaking time increases, maintaining the same level as before immersion.

Therefore, the implantable bioelectronic device of the present disclosure, which employs a gold/silver bilayer, enhances the durability of the electrode in biological environments by protecting the surface of the silver electrode with a gold electrode that provides corrosion resistance against biological fluids.

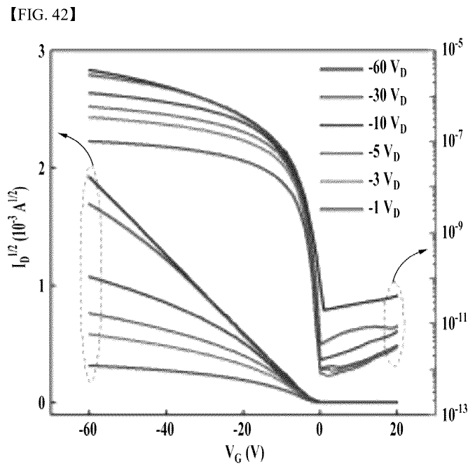

FIG. 39 is a set of graphs illustrating the XPS depth profiling results of the Ag/Au electrode and Example 4.

From FIG. 39, a mixed region of carbon and silver atoms was identified, indicating improved stretchability and adhesion.

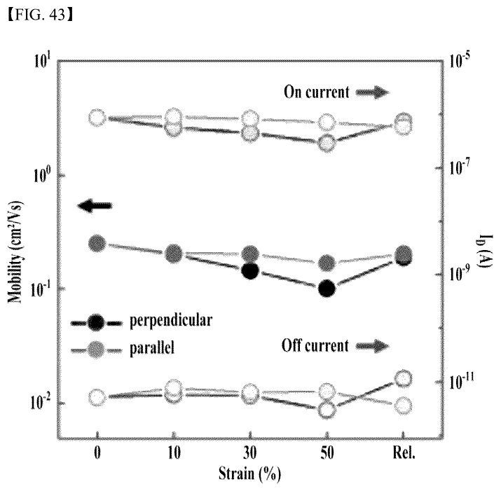

FIG. 40 is a set of graphs illustrating the transfer characteristics of the transistors of Comparative Example 1, Comparative Example 2, and Example 7, dependent upon a drain voltage.

Referring to FIG. 40, the transistor of Example 7 exhibits electrical performance similar to that of the transistor using a pure silver electrode in Comparative Example 1, while also showing improved durability in biological fluid environments.

The result showing that Example 7, which uses a gold/silver bilayer electrode, exhibits electrical performance similar to Comparative Example 1, which uses a pure silver electrode, indicates that even when a gold/silver bilayer electrode is used, the excellent electrical contact properties of silver are maintained, just as when silver is used alone.

FIG. 41 is a set of graphs illustrating the resistance measurements of Comparative Example 1, Comparative Example 2, and Example 7 before and after immersion in PBS solution, under tensile deformation ranging from 0% to 50% and in the relaxed state.

Referring to FIG. 41, the observed resistance values generally tend to increase as the tensile deformation rate increases. Before immersion in PBS, all three samples-Comparative Example 1, Comparative Example 2, and Example 7-exhibited relatively small increases in resistance. In the relaxed state of tensile deformation, the resistance values tended to recover to levels close to those before deformation.

However, after immersion in PBS solution, the trend in resistance change under tensile deformation showed significant differences.

Example 7, which uses a silver/gold bilayer electrode, maintained a resistance of approximately 80Ω even at 50% tensile deformation, similar to the resistance value before PBS immersion. In contrast, Comparative Example 2, which uses a gold electrode, exhibited a slightly higher resistance than its initial value before PBS immersion, and it was observed that even in the relaxed state after tensile deformation, the resistance did not fully return to its original level, remaining somewhat elevated.

In addition, Comparative Example 1, which uses a silver electrode, exhibited a significantly high resistance even at 10% tensile deformation, and it was observed that the high resistance was maintained even in the relaxed state after the deformation was removed.

Based on the results from FIGS. 33 to 41, it can be concluded that the stretchable transistor using the silver/gold bilayer electrode according to the present disclosure possesses high conductivity, high stretchability, and biocompatibility, making it suitable for use as a stretchable electrode for implantable bioelectronic devices.

### [Property Evaluation 2]

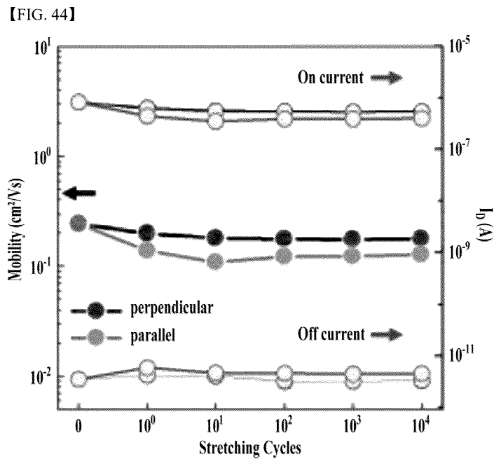

FIG. 42 is a set of graphs illustrating the transfer characteristics of Example 7 at drain voltages ranging from −1 V to −60 V.

Referring to FIG. 42, it can be seen that Example 7 operates stably even at low voltages below 10-1 V, confirming its potential for operation under low-voltage conditions, which are essential for in vivo applications.

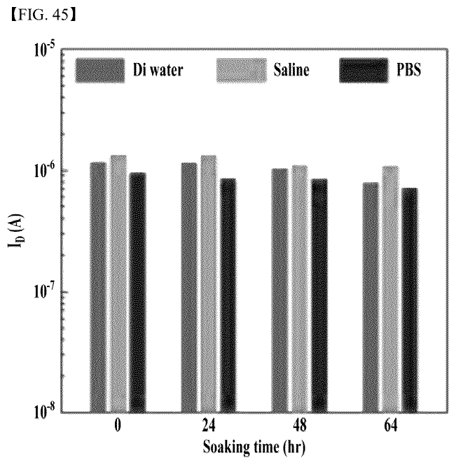

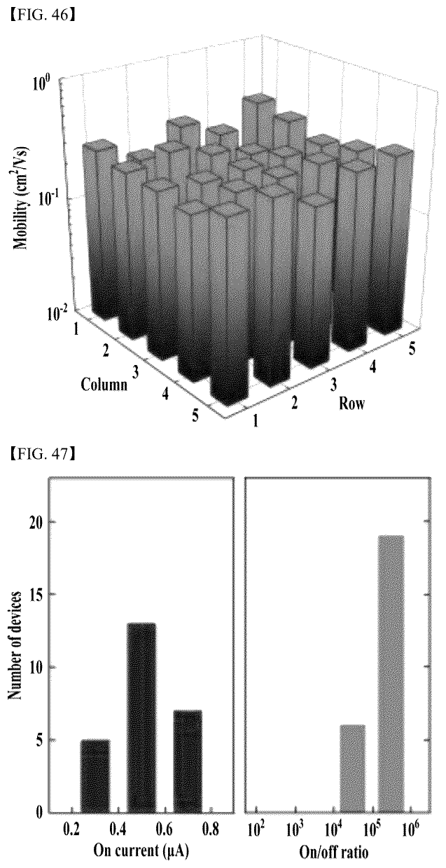

FIG. 43 is a set of graphs illustrating the field-effect mobility and on/off current ratio of Example 7 measured under tensile deformation from 0% to 50% in vertical and horizontal directions, as well as in a relaxed state. FIG. 44 illustrates the results of field-effect mobility and on/off current ratio measurements for Example 7 under multiple stretching cycles at a 50% tensile deformation condition.

Referring to FIGS. 43 and 44, it can be seen that Example 7 maintained stable electrical performance and mechanical durability under tensile deformation up to 50% in both the vertical and horizontal directions. Moreover, under a 50% tensile deformation condition, Example 7 retained its initial field-effect mobility and on/off current ratio even after up to 10,000 stretching cycles.

FIG. 45 is a set of graphs illustrating the change in drain current value after immersion in deionized water, saline solution, and PBS solution for 72 hours.

According to FIG. 45, the drain current value does not exhibit a significant change over 72 hours regardless of the type of solution, which indicates that the use of a gold/silver bilayer electrode is effective in the fabrication of a stretchable electrode for manufacturing a stretchable transistor.

### Fabrication and Property Evaluation of Active Matrix Transistor

### [Example 8] Fabrication of Active Matrix Transistor Array

Example 8 was fabricated in the same manner as Example 7, except for the method of fabricating a dielectric film. In Example 8, the dielectric film was formed using SEBS at a concentration of 75 mg/ml in toluene, and a thicker dielectric film with a thickness of 1.9 μm was obtained by spin-coating at 1000 rpm for 1 minute. The channel length and channel width were 200 μm and 150 μm, respectively.

### [Property Evaluation]

To verify the practical applicability of the present disclosure, a 5×5 active matrix array of an implantable transistor was fabricated, and the field-effect mobility, drain current, and on/off ratio were investigated.

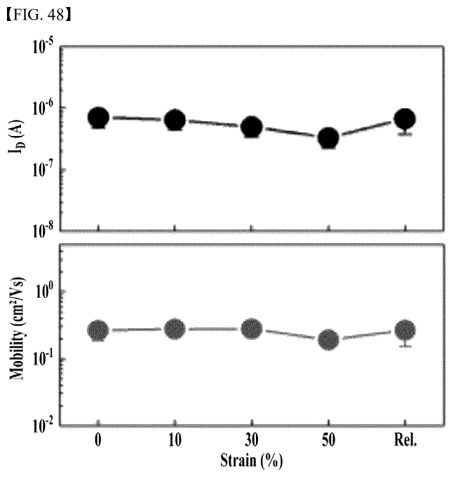

FIG. 46 illustrates the 5×5 field-effect mobility mapping results of Example 8, FIG. 47 illustrates the on-current distribution (left) and on/off ratio (right) of 25 devices in Example 8, and FIG. 48 illustrates the current (top) and field-effect mobility (bottom) of Example 8 under a tensile deformation rate ranging from 0% to 50%.

Referring to FIGS. 46 and 47, the active matrix array of Example 8 exhibited uniform field-effect mobility and drain current across all devices, confirming its high potential and suitability for bioelectronic applications.

In addition, referring to FIG. 48, it can be confirmed that the consistent field-effect mobility of Example 8 is exhibited, and changes in on-current and mobility are minimized under a tensile deformation rate ranging from 0% to 50%.

### <In-Vitro Biocompatibility Evaluation>

An in-vitro biocompatibility test was conducted using human dermal fibroblasts (hDF) and macrophages (Mφ) to evaluate the safety and efficacy of materials used in the implantable semiconductor and transistor.

The hDF cells were purchased from Lonza (Basel, Switzerland) and cultured in Dulbecco's Modified Eagle's Medium (DMEM; Gibco BRL, Gaithersburg, Maryland, USA) supplemented with 10% (v/v) fetal bovine serum (FBS; Gibco BRL) and 1% (v/v) penicillin/streptomycin (PS; Gibco BRL). During cell culture, the conditions were maintained at 37° C. in a 5% CO₂-saturated environment.

The cell culture medium was replaced every two days, and passage 8 cells were used for the experiment. Raw 264.7 cells were cultured in DMEM (Gibco BRL) supplemented with 10% (v/v) FBS and 1% (v/v) PS.

### 1. Cell Viability Assessment

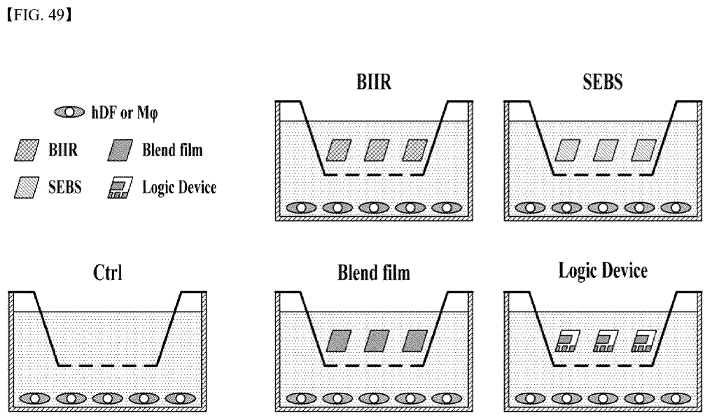

FIG. 49 illustrates the schematic overview of the cell viability assessment.

More specifically, hDF cells were seeded into a 24-well plate (2×10⁴ cells/well) and co-cultured with each material of BIIR, SEBS, the semiconductor film (blend film) of Example 4, or a logic device using a Transwell. After 1 day of incubation, cell proliferation was measured using CCK-8.

Cells were washed with PBS, and the culture medium was replaced with a medium containing 10% (v/v) CCK-8 solution. After 2 hours of incubation at 37° C., the absorbance of each well was measured at 450 nm (Infinite F50; TECAN, Männedorf, Switzerland).

Through the in vitro cell viability test shown in FIG. 49, the effects of BIIR, SEBS, the semiconductor film (blend film) of Example 4, and the logic device on cell viability, migration, and gene expression were evaluated. Live/dead assays and CCK-8 assays were used to assess cell viability.

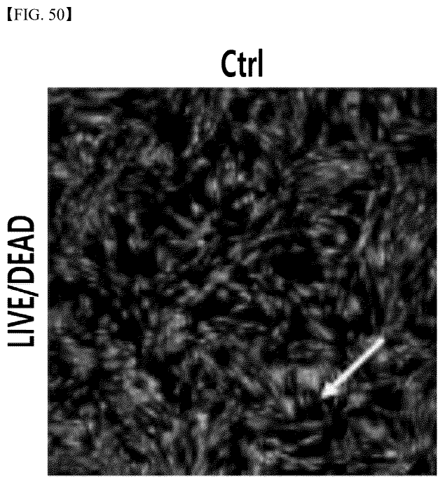

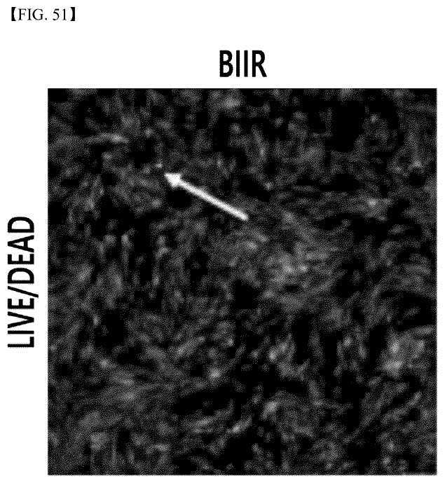

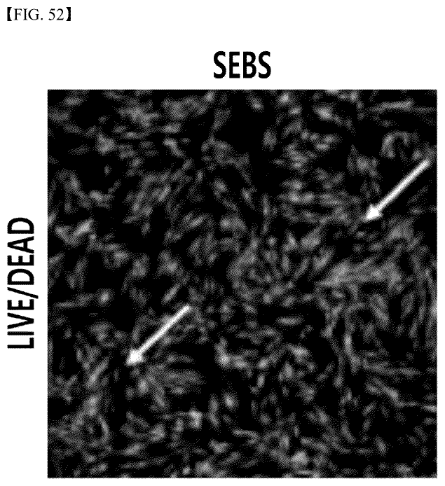

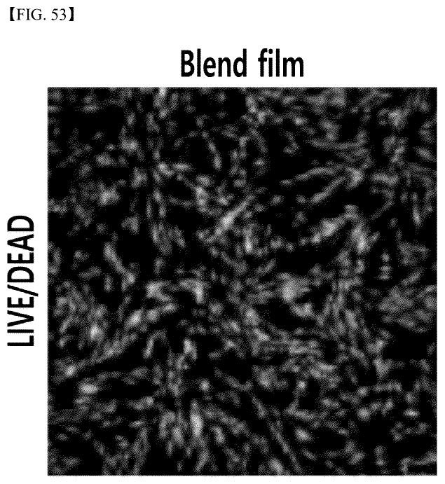

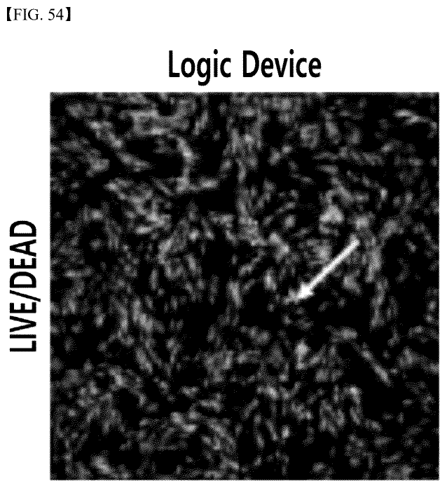

FIGS. 50 to 55 illustrate the results of the live/dead assay and the CCK-8 assay for BIIR, SEBS, the semiconductor film (blend film) of Example 4, and the logic device. More specifically, FIGS. 50 to 54 illustrate the results of the live/dead assay, and FIG. 55 illustrates the result of the CCK-8 assay.

Referring to FIGS. 50 to 54, a minimal red signal was observed in all material groups, confirming high cell viability. The CCK-8 assay results in FIG. 55 further support these findings, indicating no significant difference in cell viability among the groups.

The results of FIGS. 50 to 55 indicate that all materials used in the semiconductor film (blend film) and the logic device possess excellent biocompatibility.

### 2. In-Vitro Wound Closure Assay

hDF cells were cultured in a 6-well plate and then supplemented with DMEM (Gibco BRL). Subsequently, BIIR, SEBS, the semiconductor film (blend film) of Example 4, or the logic device was sequentially co-cultured with the cultured cells for one day, and a linear scratch was made on the hDF layer using a P1000 pipette tip.

After 24 hours of incubation, the gap width of the scratch was measured and compared with the initial gap size at time zero. The gap width of the scratch was measured from digital images using Adobe Photoshop CC (Adobe Systems, CA, USA).

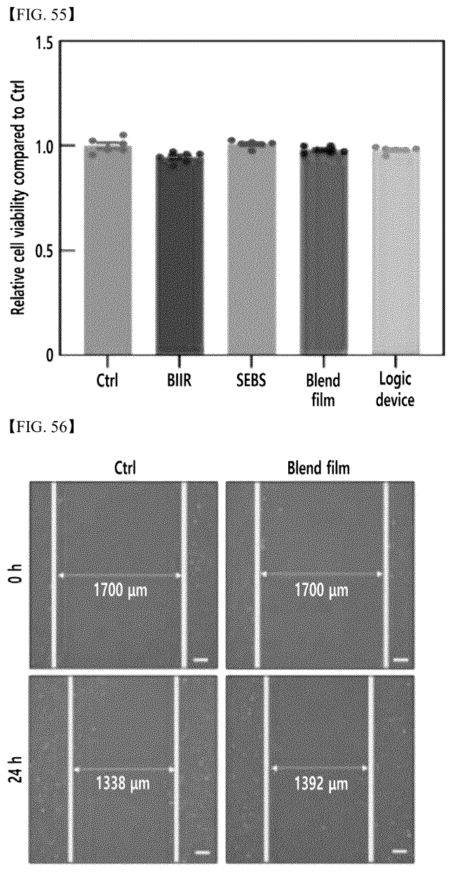

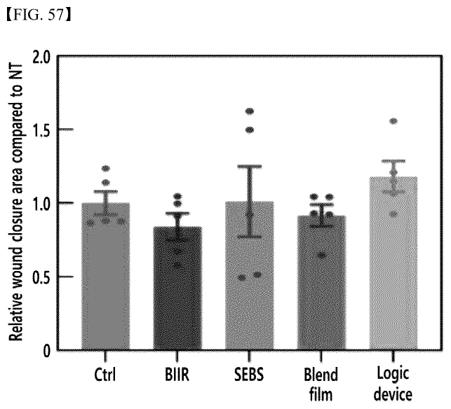

FIG. 56 illustrates in-vitro wound closure assay images in which human dermal fibroblasts (hDF) were co-cultured with the semiconductor film, and FIG. 57 illustrates graphs quantifying the number of cells in the wound area for each group after cell migration. The control group refers to samples in which no device was added to the cultured cells.

Referring to FIGS. 56 and 57, all material groups exhibited similar wound closure rates, indicating that all the materials used in the present disclosure do not impair the migratory characteristics of hDF.

### 3. Quantitative Real-Time Polymerase Chain Reaction (qRT-PCR)

The relative gene expression levels of human BCL-2, BAX, CXCR-4, PCNA, Ki67, and mouse CD80, CD86, iNOS1, iNOS2, and STAT1 were quantified using qRT-PCR. The human-specific gene primers were used for the in vitro analysis of hDF, while the mouse gene primers were used for the in vivo analysis of skin tissues.

Total ribonucleic acid (RNA) was extracted from the samples using 1 mL of TRIzol reagent (Life Technologies, Inc., Carlsbad, CA, USA) and 200 μL of chloroform, followed by centrifugation at 12,000 rpm for 10 minutes at 4° C.

The resulting RNA pellet was washed with water and 75% (v/v) ethanol, dried, and then dissolved in RNase-free water. For qRT-PCR, the SsoAdvanced™ Universal SYBR Green Supermix kit (Bio-Rad, Hercules, CA, USA) and the CFX Connect™ real-time PCR detection system (Bio-Rad) were used. Table 2 below lists the primers used for qRT-PCR.

TABLE 2

| Primers | Sequences |
|---|---|
| Human | F: 5′-GTCGGAGTCAACGGATTTGG-3′ |
| GAPDH | R: 5′-GGGTGGAATCATTGGAACAT-3′ |
| HUMAN | F: 5′-CAACATCGCCCTGTGGATGA-3′ |
| BCL-2 | R: 5′-GGGCCAAACTGAGCAGAGTC-3 |
| HUMAN | F: 5′-GCAACTTCAACTGGGGCCGGG-3′ |
| BAX | R: 5′-GATCCAGCCCAACAGCCGCTC-3′ |
| HUMAN | F: 5′-TAC ACC GAG GAA ATG GGC TCA-3′ |
| CSCR-4 | R: 5′-AGA TGA TGG AGT AGA TGG TGG G-3′ |
| HUMAN | F: 5′-AGG GCT GAA GAT AAT GCT GAT ACC-3′ |
| PCNA | R: 5′-CTC CTG TTC TGG GAT TCC AAG TTG-3′ |
| HUMAN | F: 5′-TGACCCTGATGAGAAAGCTCAA-3′ |
| Ki67 | R: 5′-CCCTGAGCAACACTGTCTTTT-3′ |
| Mouse | F: 5′-AGTTTCTCTTTTTCAGGTTGTGAA-3′ |
| CD80 | R: 5′-ACATGATGGGGAAAGCCAGG-3′ |
| Mouse | F: 5′-CTTACGGAAGCACCCACGAT-3′ |
| CD86 | R: 5′-CGGCAGATATGCAGTCCCAT-3′ |
| Mouse | F: 5′-CTGGGAGCGCTCTAGTGAAG-3′ |
| iNOS | R: 5′-CTCTCCACTGCCCCAGTTTT-3′ |
| Mouse | F: 5′-GATCGCTTGCCCAACTCTTG-3′ |
| STAT1 | R: 5′-ACTGTGACATCCTTGGGCTG-3′ |

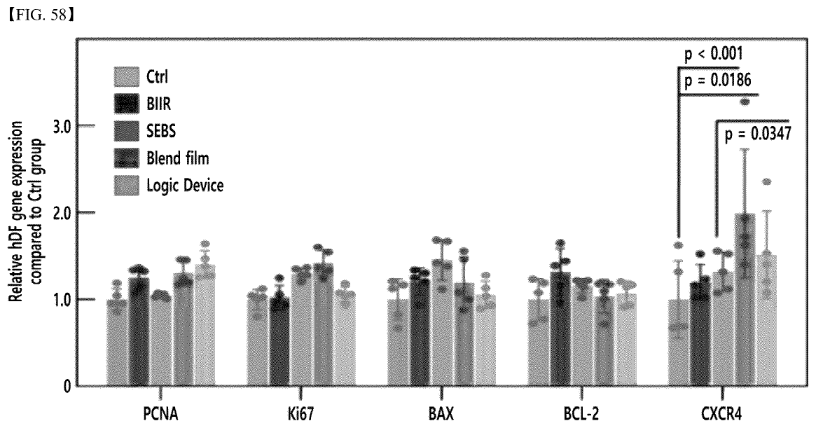

FIG. 58 illustrates the results of gene expression analysis related to proliferation, apoptosis, and migration of HDF using qRT-PCR.

Referring to FIG. 58, except for the significant increase in CXCR4 expression in Example 4, there were no notable differences in the expression of proliferation-related genes (PCNA and Ki67) and apoptosis-related genes (BAX and BCL2) among the material groups.

The increased expression of CXCR4 after using Example 4 indicates that the migratory characteristics of hDF were enhanced by Example 4.

### 4. Anti-Bacterial Test

An antibacterial test was conducted using two representative bacterial strains, namely Escherichia coli (E. coli) and Staphylococcus aureus (S. aureus), commonly associated with infections in medical devices. In this study, genetically modified E. coli C2925 and S. aureus NCTC 8325-4 strains expressing green fluorescent protein (GFP) were used.

Both bacterial strains were cultured in tryptic soy broth containing ampicillin (100 μg/mL) and chloramphenicol (10 μg/mL) at 37° C. for 24 hours, and then further cultured in fresh media until the OD600 reached 10⁸ CFU/mL. Subsequently, each strain was applied at a concentration of 5×10⁷ CFU/mL per well to the film samples prepared in 24-well plates (n=6).

Gelatin-coated (0.1% w/w) glass coverslips, which promote bacterial adhesion and growth, were used as a positive control along with bare polydimethylsiloxane (PDMS) blocks. After incubating at 37° C. for 24 hours, unattached bacteria were removed by washing three times with PBS, and the bacterial colonies were fixed with 4% paraformaldehyde (PFA) solution for 10 minutes, followed by two additional washes with PBS.

Bacteria were imaged using a confocal microscope (LSM800, Carl Zeiss AG, Germany) at 40× magnification at the BT Research Facility Center of Chung-Ang University. Z-stack images were processed using the maximum projection method, and the fluorescence intensity was measured using ImageJ (NIH, Bethesda, MD, USA) to quantify the relative number of bacteria attached to each sample.

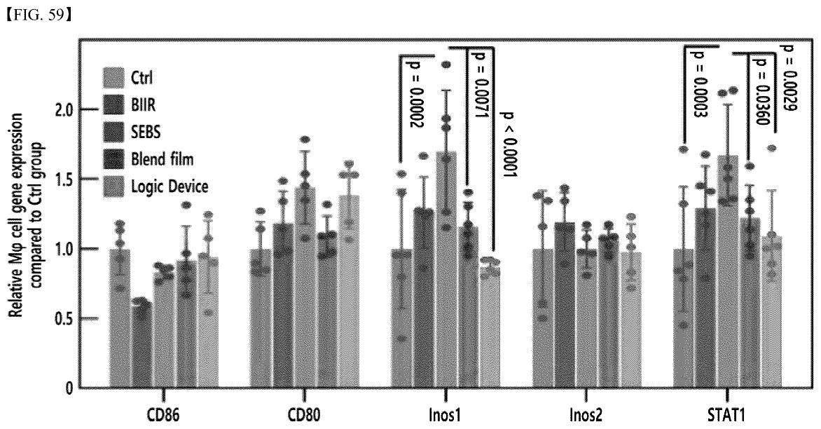

FIG. 59 is a set of graphs quantifying the relative gene expression patterns of macrophages (Mφ, Raw264.7 cells) through an antibacterial test for the material groups of BIIR, SEBS, Example 4's semiconductor film (blend film), and the logic device. The gene expression of macrophages was assessed by co-culturing each material group with the macrophages and measuring the expression of specific markers associated with pro-inflammatory (M1) macrophages.

Referring to FIG. 59, BIIR induced the lowest level of CD86 expression, and both Example 4's semiconductor film (blend film) and BIIR exhibited a balanced profile in the expression of CD80, STAT1, and iNOS compared to the control group.

In contrast, the SEBS group showed the highest levels of gene expression, indicating a higher potential to induce inflammation.

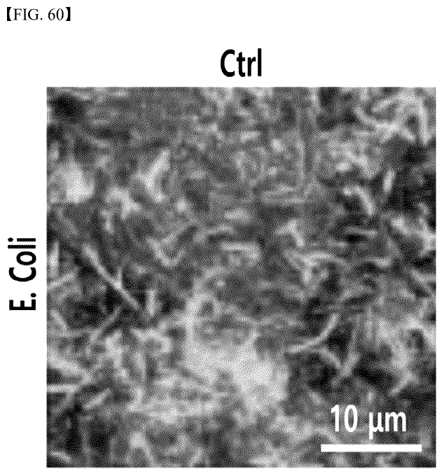

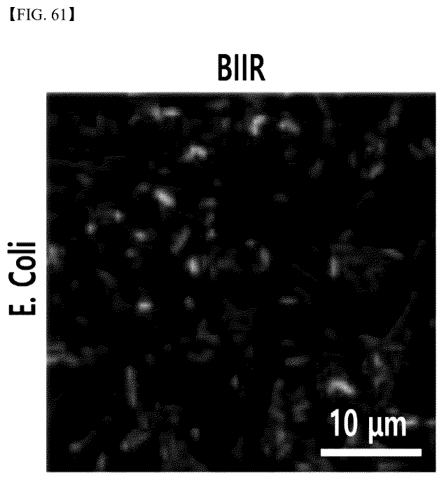

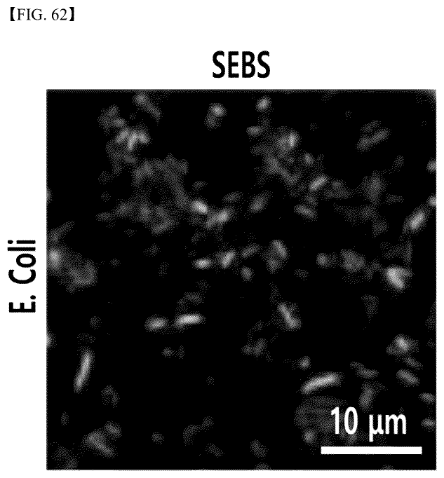

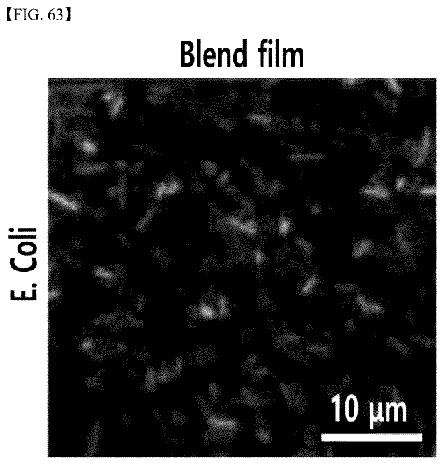

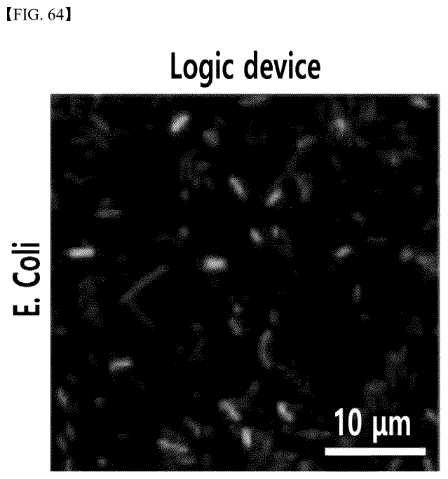

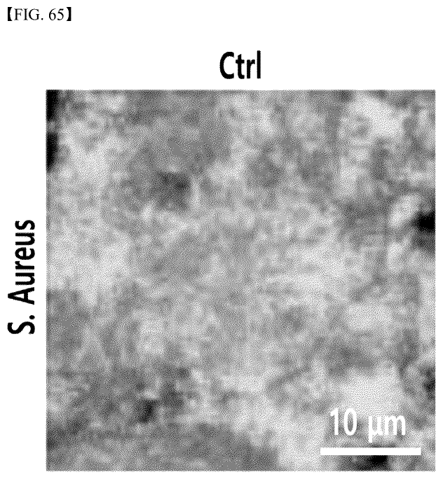

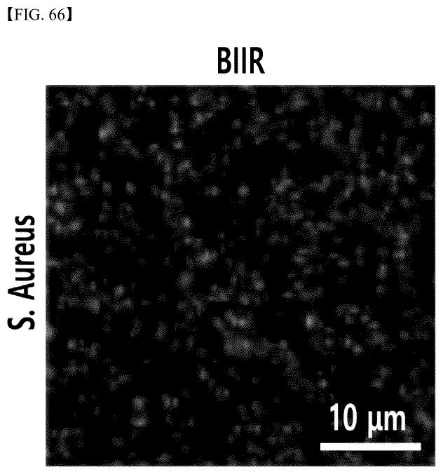

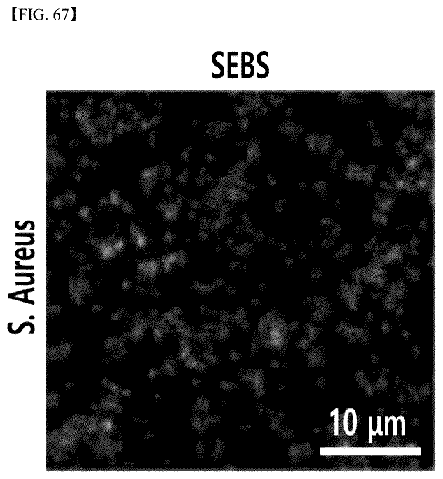

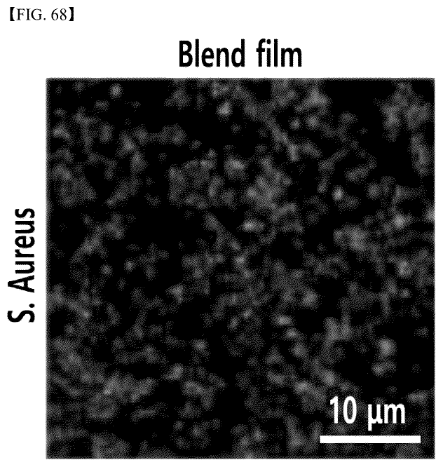

FIGS. 60 to 69 illustrate green fluorescence images of genetically modified Escherichia coli (E. coli, top) and Staphylococcus aureus (S. aureus, bottom) for each material group of BIIR, SEBS, Example 4's semiconductor film (blend film), and logic device. FIGS. 70 and 71 illustrate graphs quantifying the cell viability of genetically modified E. coli (top) and S. aureus (bottom) for the same material groups.

Referring to FIGS. 60 to 71, the positive control (gelatin-coated glass coverslip) exhibited significantly high bacterial growth, whereas BIIR, SEBS, Example 4's semiconductor film (blend film), and the logic device showed no significant difference in the number of bacteria across material groups, indicating antibacterial properties.

As shown in FIGS. 60 to 71, the stretchable semiconductor film and logic device with biocompatibility according to the present disclosure do not induce inflammatory responses, exhibit high cell viability, enable cell proliferation and migration, and demonstrate enhanced antibacterial effects, suggesting their potential application as medical devices.

### In-Vivo Biocompatibility Test for Implantable Transistor

### 1. Implantation of Semiconductor Film and Logic Device

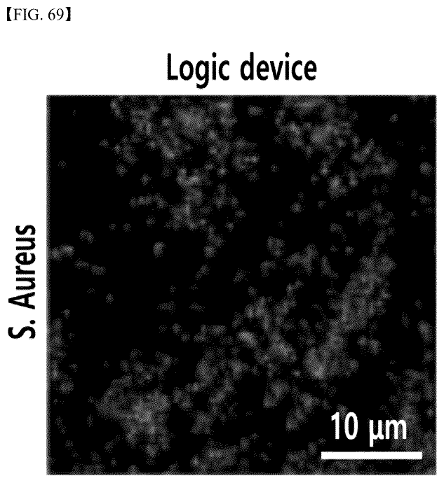

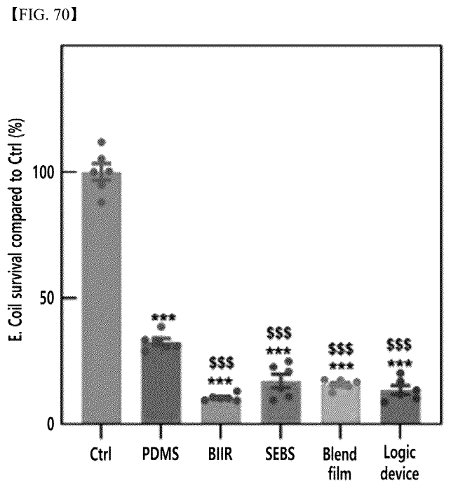

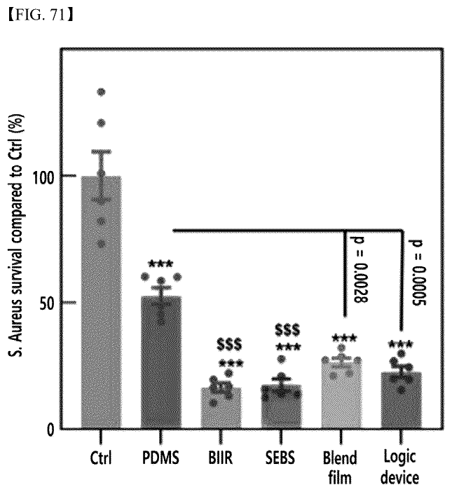

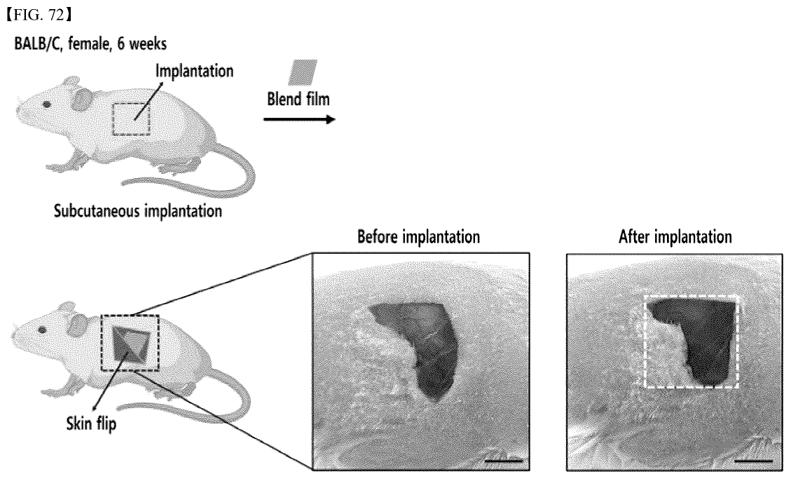

FIG. 72 is a schematic diagram illustrating the experimental method for evaluating the in-vivo biocompatibility of the stretchable semiconductor film having biocompatibility according to the present disclosure, using a subcutaneous implant model in BALB/C mice. The goal was to assess long-term inflammatory responses, tissue integration, and overall compatibility of the material.

More specifically, 6-week-old female Balb/C mice (body weight 20-25 g; Orient, Seoul, Republic of Korea) were anesthetized with 200 μL of xylazine (20 mg/kg) and ketamine (100 mg/kg) diluted in saline. Hair was removed from the right dorsal area using an electric animal clipper and a razor (less than 1 minute).

The implantation site was marked with a 10×10 mm² stamp on the lateral flank of each mouse. The epidermis, dermis, and stratum corneum were surgically incised from the upper and right side, and the skin was flipped open.

For the semiconductor film and logic device groups, each material was implanted in the opened area, and the same surgical procedure was performed on the sham group without implantation. At 3 and 30 days post-implantation, the subcutaneously implanted mice were sacrificed for in vivo analysis. All animals were maintained in accordance with the Animal Care and Use Guidelines of Sungkyunkwan University (SKKUIACUC2022-04-40-1).

### 2. Western Blotting

The harvested tissues were homogenized and lysed in RIPA buffer (Rockland Immunochemicals, Inc., Limerick, PA, USA). The lysate was centrifuged at 10,000 g for 10 minutes, and the supernatant was collected as the protein extract.

The protein concentration was determined using the BCA assay (Pierce Biotechnology, Rockford, IL, USA). Equal amounts of protein from each sample were mixed with sample buffer and subjected to sodium dodecyl sulfate-polyacrylamide gel electrophoresis (SDS-PAGE) using a 10% (v/v) resolving gel.

The separated proteins were transferred to an immuno-blot PVDF membrane (Bio-Rad). The membrane was blocked in Tris-buffered saline with Tween 20 (TBS-T; 50 mM Tris-HCl (pH 7.5), 150 mM NaCl, 2.5 mM KCl) containing 5% (w/v) non-fat dry milk at 25° C. for 1 hour.

The membrane was then incubated overnight at 4° C. with antibodies against glyceraldehyde 3-phosphate dehydrogenase (GAPDH) (Abcam, ab9485, Cambridge, UK), CD163 (Abcam, ab182422), CD206 (Abcam, ab64693), STAT1 (Cell Signaling Technology, CS9172), STAT6 (Cell Signaling Technology, CS9362), and TGF-β (Cell Signaling Technology, CS3711).

Next, the membrane was incubated at 25° C. for 1 hour with horseradish peroxidase (HRP)-conjugated secondary antibodies (R&D Systems, HAF008 for GAPDH; HAF017 for CD163, CD206, STAT1, STAT6, and TGF-β; Minneapolis, MN, USA), followed by treatment with ECL reagent (TransLab, Daejeon, Republic of Korea). The blot was developed in a darkroom, and the chemiluminescent signals were captured using X-ray blue film (Agfa HealthCare NV, Mortsel, Belgium).

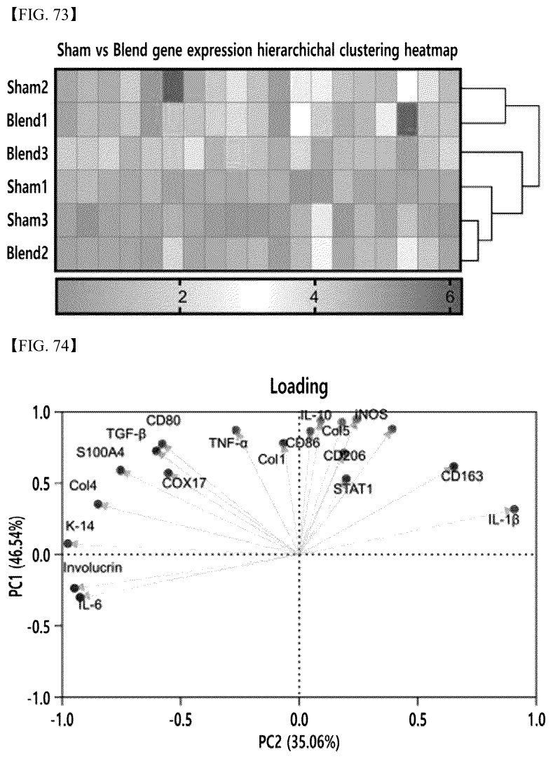

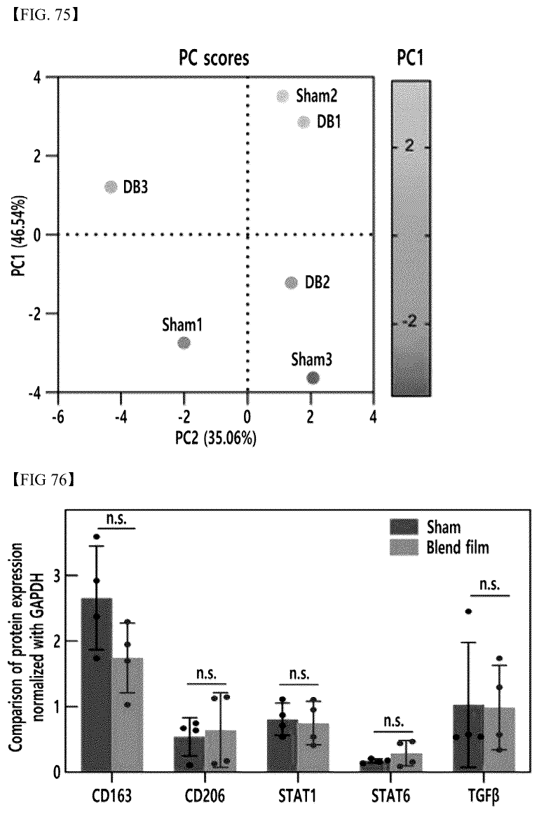

FIG. 73 illustrates a hierarchical clustering heatmap of gene expression in the sham group and the semiconductor film implantation group, FIG. 74 illustrates the distribution of gene expression levels measured in both groups, and FIG. 75 illustrates the principal component analysis (PCA) scores based on gene expression measurements for the two groups.

Referring to FIG. 73, the hierarchical clustering heatmap analysis shows no significant difference between the sham group and the implantation group. Principal component analysis (PCA) performed on the gene data including 19 genes per group identified five principal components, with PC1 accounting for 46.54% of the variance (eigenvalue: 8.843) and PC2 accounting for 35.06% (eigenvalue: 6.660).

Additionally, as shown in FIG. 74, IL-10 and iNOS (positive direction) and IL-6 (negative direction) significantly influence PC1, whereas IL-1B (positive direction) and K-14 (negative direction) have a notable impact on PC2.

Furthermore, referring to FIG. 75, there is no significant difference in the distribution of PC scores between the sham group and the semiconductor film implantation group, indicating that the stretchable semiconductor film having biocompatibility according to the present disclosure is compatible with biological tissues and has potential for long-term implantation.

FIG. 76 is a graph comparing macrophage phenotype markers between the sham group and the semiconductor film implantation group, based on Western blot analysis.

According to FIG. 76, the expression levels of both M1 and M2 macrophage markers were similar in the sham and implantation groups, indicating that no severe inflammation was observed, which in turn suggests that tissue homeostasis was maintained.

### 3. Hematoxylin and Eosin Staining Analysis (H&E)

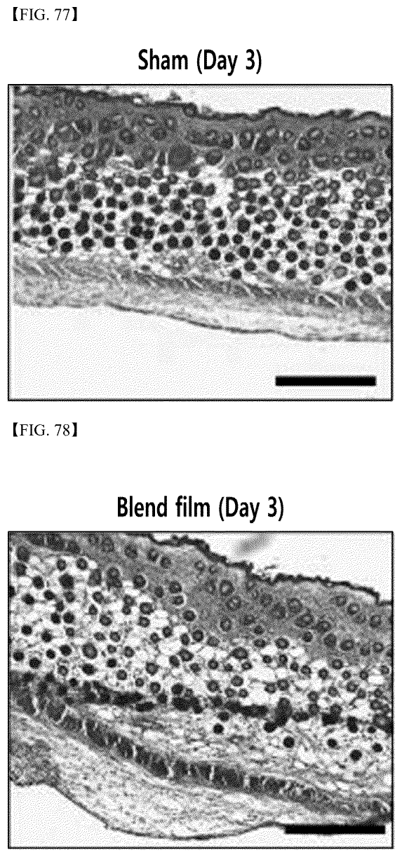

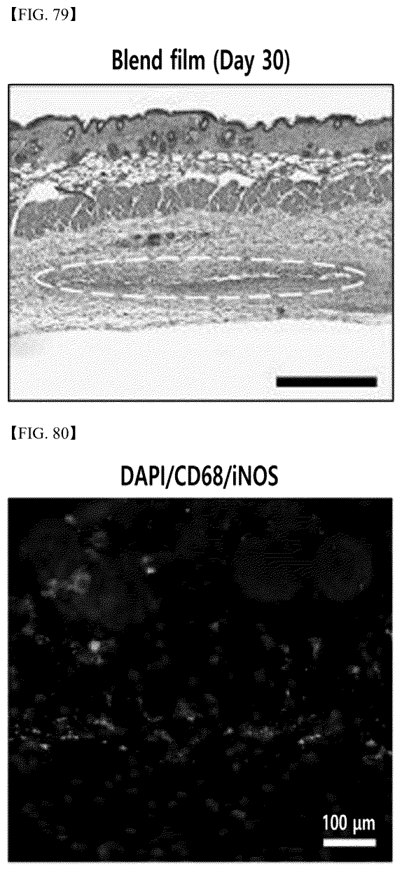

FIGS. 77 to 79 illustrate the hematoxylin and eosin (H&E) staining results of skin tissues harvested from the implantation sites of the sham group and the semiconductor film implantation group.

According to FIGS. 77 to 79, the tissue sections surrounding the implantation sites exhibited normal collagen layer formation, and even after 30 days post-implantation, the implanted group showed a tissue structure similar to that of the control group. These results suggest that the stretchable semiconductor film having biocompatibility according to the present disclosure integrates well with the host tissue without adverse effects.

FIGS. 80 to 83 illustrate the immunohistochemical staining results for M1 and M2 macrophage markers in the sham group and the semiconductor film implantation group.

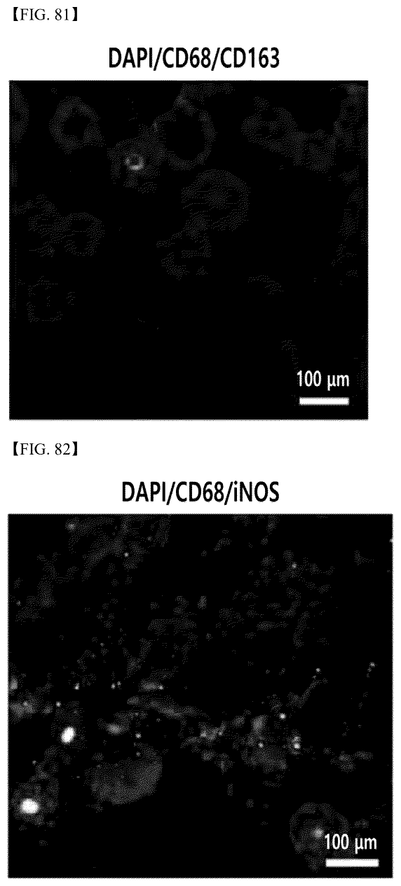

More specifically, FIG. 80 illustrates the immunohistochemical staining result for M1 macrophage marker in the sham group, FIG. 81 for M2 macrophage marker in the sham group, FIG. 82 for M1 macrophage marker in the DPPT-TT: BIIR group, and FIG. 83 for M2 macrophage marker in the DPPT-TT: BIIR group.

In FIGS. 80 to 83, iNOS is the M1 marker, CD163 is the M2 marker, and CD68 is the macrophage (Mφ) marker.

As shown in FIGS. 80 to 83, the stained sections for iNOS (M1 marker), CD163 (M2 marker), and CD68 (Mφ marker) exhibited no significant differences between the sham group and the implantation group, indicating that implantation of the stretchable semiconductor film having biocompatibility according to the present disclosure does not induce an inflammatory response.

### <Property Evaluation of Implantable Logic Circuit>

To demonstrate the practical application of implantable transistors in bioelectronic devices such as skin, we developed various logic circuits, including an inverter, a NOR, and a NAND gate, and conducted in-vivo testing.

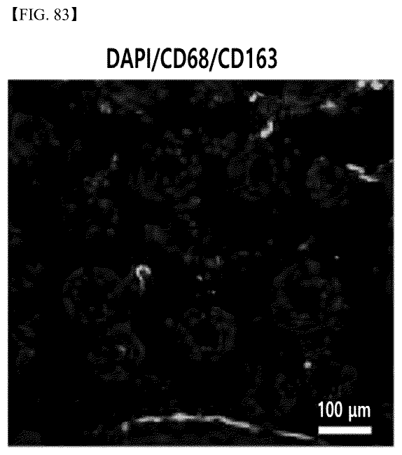

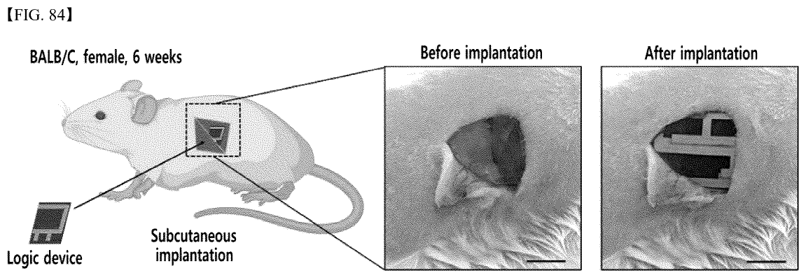

FIG. 84 is the schematic diagram of the subcutaneous implantation of a logic circuit fabricated with an implantable transistor. This circuit was designed to maintain stable electrical performance under mechanical deformation and physiological conditions. In the present disclosure, to verify the functionality of the logic circuit in vivo, the device was implanted in BALB/C mice, and its performance was monitored for 3 days.

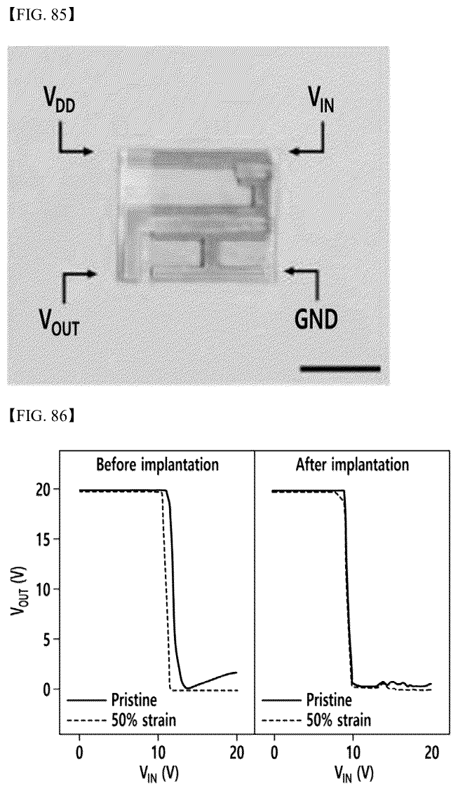

FIG. 85 illustrates a photograph of an inverter circuit based on CMOS design, FIG. 86 illustrates the voltage transfer characteristics (VTC) of an inverter under 0% strain (left) and 50% strain (right) before and after implantation in a mouse, and FIG. 87 illustrates the gain of the inverter under the same strain conditions before and after implantation.

According to FIGS. 86 and 87, the inverter circuit exhibited clear switching behavior and stable voltage transfer characteristics (VTC) even under 50% strain, and the gain of the inverter remained consistently maintained after implantation and 50% tensile deformation, indicating mechanical robustness and stability in a biological environment.

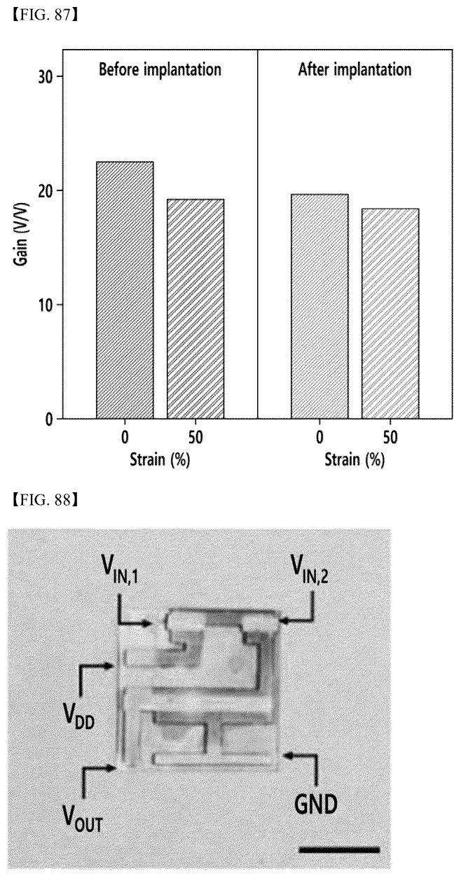

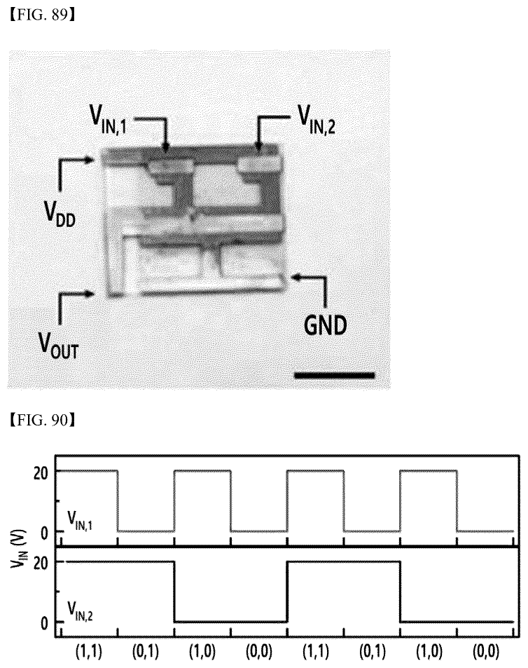

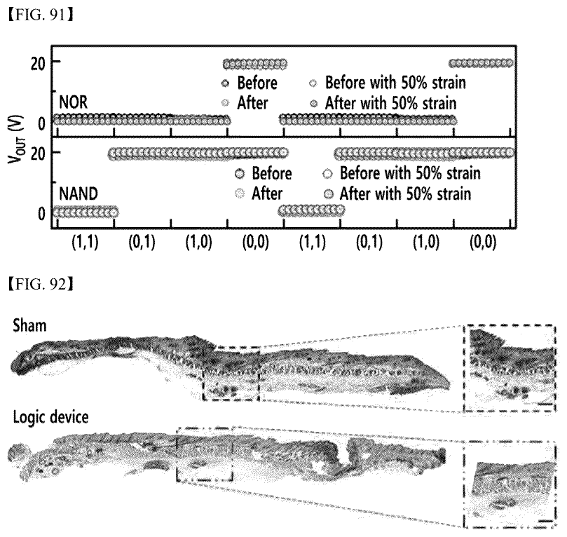

FIGS. 88 and 89 illustrate photographs of NOR and NAND circuits based on CMOS design, and FIGS. 90 and 91 illustrate graphs analyzing the input and output characteristics of the NOR and NAND devices under 0% strain and 50% strain, before and after implantation in mice.

More specifically, FIG. 88 illustrates a photograph of the NOR circuit, FIG. 89 shows that of the NAND circuit, FIG. 90 illustrates the input characteristics, and FIG. 91 shows the output characteristics.

According to FIGS. 90 and 91, the NOR and NAND gates maintained consistent logical output states even after implantation and 50% tensile deformation. Therefore, the implantable logic circuit of the present disclosure may maintain stable electronic operation under biological conditions and mechanical deformation.

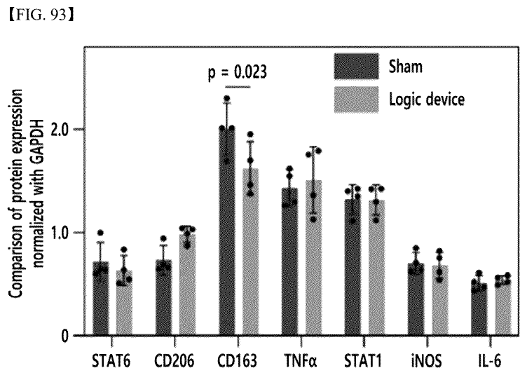

FIG. 92 illustrates a tissue image of the logic circuit implantation site, and FIG. 93 illustrates a graph quantifying the Western blot bands for M1 and M2 macrophage markers and inflammation-related markers.

According to FIGS. 92 and 93, short-term (3-day) implantation resulted in no immune cell infiltration in both the sham group and the logic circuit implantation group, and all markers, except for CD163, showed similar Western blot band intensities between the sham group and the implanted group.

As shown in FIGS. 92 and 93, the implantable logic circuit of the present disclosure does not induce significant inflammation, suggesting that it may function effectively in a biological environment.

The present disclosure can provide a semiconductor film with enhanced stretchability by forming a phase-separated, interconnected nanofiber network in which a semiconducting polymer is embedded within an elastomer matrix.

The present disclosure can provide a semiconductor film with improved mechanical and electrical properties by subjecting the elastomer matrix to vulcanization and chemical cross-linking.

The present disclosure can provide an implantable semiconductor film that exhibits stretchability, corrosion resistance, and biocompatibility in a biological environment, by embedding a semiconducting polymer in a biocompatible elastomer matrix.

The present disclosure can provide a stretchable electrode for an implantable bioelectronic device by vapor-depositing a metal electrode onto a stretchable implantable semiconductor film.

The present disclosure can provide an implantable bioelectronic device with enhanced corrosion resistance, flexibility, stretchability, and biocompatibility in biological environments by using a bilayer electrode composed of gold and silver.

Although the present disclosure has been described through limited examples and drawings, the present disclosure is not intended to be limited to the examples. Those skilled in the art will appreciate that various modifications, additions and substitutions are possible, without departing from the scope and spirit of the disclosure. Therefore, the scope of the present disclosure should not be limited to the described examples, but should be defined not only by the claims described below but also by equivalents of these claims.

### DESCRIPTION OF SYMBOLS

100: stretchable semiconductor film having biocompatibility
110: semiconducting polymer
120: elastomer matrix
230: crosslinking agent
300: implantable bioelectronic device
310: substrate
320: gate electrode
330: gate insulating layer
340: stretchable semiconductor layer having biocompatibility
350: source electrode
360: drain electrode

What is claimed is:

1. A stretchable semiconductor film having biocompatibility, comprising:
an elastomer matrix having biocompatibility; and
a semiconducting polymer of an interconnected semiconductor nanofiber network embedded in the elastomer matrix,
wherein the interconnected semiconductor nanofiber network of the semiconducting polymer is formed through phase separation due to a difference in surface energy between the semiconducting polymer and the elastomer matrix during a mixing process, and
the elastomer matrix is vulcanized and, accordingly, chemically cross-linked.

2. The stretchable semiconductor film according to claim 1, wherein the interconnected nanofiber network structure of the semiconducting polymer formed in the elastomer matrix provides stretchability.

3. The stretchable semiconductor film according to claim 1, wherein, when tensile-deformed due to the vulcanization, the interconnected semiconductor nanofiber network is maintained.

4. The stretchable semiconductor film according to claim 1, wherein a mixing ratio of the semiconducting polymer to the elastomer matrix is from 9:1 to 3:7.

5. The stretchable semiconductor film according to claim 4, wherein a field-effect mobility of the stretchable semiconductor film having biocompatibility according to the mixing ratio is maintained in a range of 5.0×10⁻¹ cm²/V·s to 5.5×10⁻¹ cm²/V·s.

6. The stretchable semiconductor film according to claim 1, wherein the elastomer matrix is bromo isobutylene isoprene rubber (BIIR).

7. An implantable bioelectronic device, comprising:
a substrate;
a gate electrode formed on the substrate;
a gate insulating layer formed on the gate electrode;
a stretchable semiconductor layer having biocompatibility formed on the gate insulating layer; and
a source electrode and drain electrode formed to be spaced apart from each other on the stretchable semiconductor layer,
wherein the gate electrode, the source electrode, and the drain electrode are configured as a bilayer electrode composed of gold and silver,
the stretchable semiconductor layer having biocompatibility comprises:
an elastomer matrix having biocompatibility; and
a semiconducting polymer of an interconnected semiconductor nanofiber network embedded in the elastomer matrix,
wherein the semiconducting polymer of the interconnected semiconductor nanofiber network is formed by phase separation due to a difference in surface energy between the semiconducting polymer and the elastomer matrix during mixing of the semiconducting polymer and the elastomer matrix, and
the elastomer matrix is vulcanized and, accordingly, chemically cross-linked.

8. The implantable bioelectronic device according to claim 7, wherein the bilayer electrode prevents corrosion in biological environments.

9. The implantable bioelectronic device according to claim 7, wherein a resistance of the implantable bioelectronic device using the bilayer electrode is maintained in a range of 0.5Ω to 2Ω in a biological environment.

10. The implantable bioelectronic device according to claim 7, wherein field-effect mobility of the implantable bioelectronic device under multiple stretching conditions is maintained in a range of 1.3×10⁻¹ cm²/V·s to 2.3×10⁻¹ cm²/V·s.
